(19) United States
(12) Patent Application Publication
(10) Pub. No.: US 2026/0031231 A1
(43) Pub. Date: Jan. 29, 2026

(54) TECHNIQUES FOR CANCER DETECTION USING NUCLEIC ACID FRAGMENTATION SITE CONTEXTS

(71) Applicant: Blackjack Biotechnologies, Inc., Baltimore, MD (US)

(72) Inventor: Derrick Wood, Baltimore, MD (US)

(21) Appl. No.: 19/277,903

(22) Filed: Jul. 23, 2025

Related U.S. Application Data
(60) Provisional application No. 63/675,236, filed on Jul. 24, 2024.

(51) Int. Cl. G16H 50/20; G16B 30/10; G16H 20/10; G16H 50/70

(52) U.S. Cl. CPC G16H 50/20; G16B 30/10; G16H 20/10; G16H 50/70

(57) ABSTRACT

Some embodiments provide techniques determining whether a subject has cancer by analyzing fragmentation in a cell-free deoxyribonucleic acid (cfDNA) sample obtained from the subject. The system identifies fragmentation sites of the cfDNA sample and corresponding fragmentation site contexts. The system generates, using the fragmentation site contexts, a data structure encoding information about a fragmentation site context distribution of the cfDNA sample. The system uses the data structure to determine whether the subject has cancer. If the cfDNA sample is found to be cancerous, the system may further use the data structure to determine a tissue of origin of the cancer.

### RELATED APPLICATIONS

This application claims benefit under 35 U.S.C. § 119(e) of U.S. Provisional Application No. 63/675,236 filed Jul. 24, 2024, and titled “TECHNIQUES FOR CANCER DETECTION USING NUCLEIC ACID FRAGMENTATION SITE CONTEXTS,” which is incorporated by reference herein.

### BACKGROUND

Cancer screening may be performed on subjects to check for the presence of abnormal cells that are cancerous or may become cancerous. Early detection of cancer in a subject can significantly improve the likelihood that the subject survives the cancer with appropriate intervention. Cancer detected in an early stage may also be more easily treated than in a later stage.

One type of cancer screening that may be performed on a subject involves performing genetic tests on deoxyribonucleic acid (DNA) from the subject. A cancer tumor sheds DNA from dying cells that then circulates in a subject's bloodstream. A blood sample may be obtained from the subject and DNA present in the sample may be analyzed using genetic testing to detect the presence of cancer. For example, an analysis may be performed on the DNA present in the sample in order to detect the presence of cells that are cancerous or may become cancerous.

### SUMMARY

Some embodiments provide techniques for determining whether a subject has cancer by analyzing fragmentation in a cell-free deoxyribonucleic acid (cfDNA) sample obtained from the subject. The system identifies fragmentation sites of a cell-free deoxyribonucleic acid (cfDNA) sample obtained from a subject and corresponding fragmentation site contexts. The system generates, using the fragmentation site contexts, a data structure encoding information about a fragmentation site context distribution of the cfDNA sample. The system uses the data structure to determine whether the subject has cancer. If the cfDNA sample is found to be cancerous, the system may further use the data structure to determine an issue of origin of the cancer.

Some embodiments provide a method for determining whether a subject has cancer by analyzing fragmentation in a cell-free deoxyribonucleic acid (cfDNA) sample obtained from the subject. The method comprises using at least one computer hardware processor to perform: accessing sequencing data, the sequencing data previously obtained from sequencing the cfDNA sample, the sequencing data comprising a plurality of reads of cfDNA fragments; aligning the plurality of reads to a reference; identifying, using results of aligning the plurality of reads to the reference, fragmentation sites of the cfDNA sample and nucleotide subsequences of the reference corresponding to the fragmentation sites; determining fragmentation site contexts of the cfDNA sample using the nucleotide subsequences of the reference corresponding to the fragmentation sites; generating, using the plurality of fragmentation site contexts, a data structure encoding information about a distribution of fragmentation site contexts of the cfDNA sample; and determining whether the subject has cancer using the data structure encoding information about the distribution of fragmentation site contexts of the cfDNA sample.

Some embodiments provide a system for determining whether a subject has cancer by analyzing fragmentation in a cell-free deoxyribonucleic acid (cfDNA) sample obtained from the subject. The system comprises a non-transitory computer-readable storage medium storing instructions and at least one computer hardware processor. The instructions, when executed by the at least one computer hardware processor cause the at least one computer hardware processor to perform: accessing sequencing data, the sequencing data previously obtained from sequencing the cfDNA sample, the sequencing data comprising a plurality of reads of cfDNA fragments; aligning the plurality of reads to a reference; identifying, using results of aligning the plurality of reads to the reference, fragmentation sites of the cfDNA sample and nucleotide subsequences of the reference corresponding to the fragmentation sites; determining fragmentation site contexts of the cfDNA sample using the nucleotide subsequences of the reference corresponding to the fragmentation sites; generating, using the plurality of fragmentation site contexts, a data structure encoding information about a distribution of fragmentation site contexts of the cfDNA sample; and determining whether the subject has cancer using the data structure encoding information about the distribution of fragmentation site contexts of the cfDNA sample.

Some embodiments provide a non-transitory computer-readable storage medium storing instructions. The instructions, when executed by at least one computer hardware processor, cause the at least one computer hardware processor to perform a method for determining whether a subject has cancer by analyzing fragmentation in a cell-free deoxyribonucleic acid (cfDNA) sample obtained from the subject. The method comprises using at least one computer hardware processor to perform: accessing sequencing data, the sequencing data previously obtained from sequencing the cfDNA sample, the sequencing data comprising a plurality of reads of cfDNA fragments; aligning the plurality of reads to a reference; identifying, using results of aligning the plurality of reads to the reference, fragmentation sites of the cfDNA sample and nucleotide subsequences of the reference corresponding to the fragmentation sites; determining fragmentation site contexts of the cfDNA sample using the nucleotide subsequences of the reference corresponding to the fragmentation sites; generating, using the plurality of fragmentation site contexts, a data structure encoding information about a distribution of fragmentation site contexts of the cfDNA sample; and determining whether the subject has cancer using the data structure encoding information about the distribution of fragmentation site contexts of the cfDNA sample.

The foregoing summary is non-limiting.

### BRIEF DESCRIPTION OF DRAWINGS

Various aspects and embodiments will be described with reference to the following figures. It should be appreciated that the figures are not necessarily drawn to scale. Items appearing in multiple figures are indicated by the same or a similar reference number in all the figures in which they appear.

FIG. 1A illustrates an example environment in which a cancer detection system may operate, according to some embodiments of the technology described herein may operate.

FIG. 1B shows the interaction between the modules of the cancer detection system of FIG. 1A, according to some embodiments of the technology described herein.

FIG. 2A illustrates identification of a fragmentation site context corresponding to a fragmentation site, according to some embodiments of the technology described herein.

FIG. 2B illustrates identification of a fragmentation site context corresponding to a fragmentation site, according to some embodiments of the technology described herein.

FIG. 3 illustrates an example of identifying fragmentation site contexts corresponding to two fragmentation sites of a cfDNA fragment, according to some embodiments of the technology described herein.

FIG. 4 shows an example data structure that may encode information about a fragmentation site context distribution of a cfDNA sample, according to some embodiments of the technology described herein.

FIG. 5A illustrates classifications that may be determined by a cancer detection system using one or more trained machine learning models, according to some embodiments of the technology described herein.

FIG. 5B illustrates determination of distance measurements using a data structure encoding information about fragmentation site context distribution of a cfDNA sample, according to some embodiments of the technology described herein.

FIG. 6 illustrates an example process for determining whether a subject has cancer by analyzing fragmentation in a cfDNA sample obtained from the subject, according to some embodiments of the technology described herein.

FIG. 7 shows a graph of mean distance between original probability mass function (PMF)/position probability matrices (PPMs) and PMF/PPMs generated using fragmentation site contexts identified using reads of cfDNA fragments according to some embodiments of the technology described herein.

FIG. 8A shows graphs illustrating distances of non-cancerous PPMs from a centroid of the non-cancerous PPMs and distances of cancerous PPMs from a centroid of the cancerous PPMs for two different datasets according to some embodiments of the technology described herein.

FIG. 8B shows another set of graphs illustrating distances of noncancerous PPMs to a distribution of the noncancerous PPMs and distances of cancerous PPMs to a distribution of the cancerous PPMs according to some embodiments of the technology described herein.

FIG. 8C shows another set of graphs illustrating distances of noncancerous PPMs to a distribution of the noncancerous PPMs and distances of cancerous PPMs to a distribution of the cancerous PPMs according to some embodiments of the technology described herein.

FIG. 9A shows a graph illustrating total class similarity (TCS) transformation of PPMs generated from samples in the HCC dataset according to some embodiments of the technology described herein.

FIG. 9B shows a graph of TCS transformation of PPMs generated from samples in the MC dataset according to some embodiments of the technology described herein.

FIG. 10A shows a graph of class Mahalanobis similarity (CMS) transformation of PPMs generated from samples in the HCC dataset according to some embodiments of the technology described herein.

FIG. 10B shows a graph of CMS transformation of PPMs generated from samples in the MC dataset according to some embodiments of the technology described herein.

FIG. 11 shows a graph illustrating use of a combination of TCS and CMS transformations (referred to as “TCMS”) to perform classification on samples in the HCC dataset, and a graph illustrating use of TCMS to perform classification on samples in the MC dataset, according to some embodiments of the technology described herein.

FIG. 12 shows a graph illustrating performance of tissue of origin classification using TCMS according to some embodiments of the technology described herein.

FIG. 13A shows a graph illustrating performance of tissue of origin classification on the MC dataset using Mahalanobis similarity according to some embodiments of the technology described herein.

FIG. 13B shows a graph comparing the performance of tissue of origin classification of an example embodiment of the technology described herein to another technique.

FIG. 14 shows a graph illustrating cancer detection using fragmentation site contexts corresponding to transcription factor binding sites (TFBSs), according to some embodiments of the technology described herein.

FIG. 15 shows a graph illustrating scores generated for healthy samples by processing fragmentation site contexts for the healthy samples using a trained machine learning model, according to some embodiments of the technology described herein.

FIG. 16 illustrates graphs showing the impact of added noise on sensitivity determined for synthetic 2D data generated using a random number generator, according to some embodiments of the technology described herein.

FIG. 17 illustrates graphs showing healthy centroid distances determined for PPMs generated from samples in various datasets, according to some embodiments of the technology described herein.

FIG. 18 shows a block diagram of an exemplary computing device, in accordance with some embodiments of the technology described herein.

### DETAILED DESCRIPTION

The inventors have developed improved computational techniques for determining whether a subject has cancer by analyzing fragmentation in a cell-free DNA (cfDNA) sample obtained from the subject. The system identifies fragmentation site contexts of the cfDNA sample and generates a data structure encoding information about the fragmentation site context distribution of the cfDNA sample. The system uses the data structure to determine whether the subject has cancer. In some embodiments, the system may determine a tissue-of-origin of cancerous cells using the data structure.

Fragmentomics involves analyzing the fragmentation of a cfDNA sample from a subject. Fragmentomics may be used to perform cancer detection in a subject. For example, a blood sample obtained from a subject that has cancer may include cfDNA fragments from cancerous cells that have genetic differences from healthy cell tissues. The cfDNA sample is sequenced to obtain reads of the cfDNA fragments which can be used to determine whether a subject has cancer. Some fragmentomics techniques involve determining whether a fragmentation pattern of the cfDNA determined using the reads indicates that the subject has cancer. For example, the fragmentation pattern of the cfDNA may be encoded as a numerical vector. The numerical vector may be compared to numerical vectors encoding fragmentation patterns associated with healthy and/or cancerous cfDNA samples to determine whether the subject has cancer. As another example, the fragmentation pattern of the cfDNA may be described by one or more parameters that are based on lengths of the reads (e.g., a ratio of short-to-long DNA fragments). The parameter value(s) may be used to determine whether the subject has cancer (e.g., by comparing parameter value(s) to parameter value(s) associated with healthy and/or cancerous cfDNA samples).

One way to characterize the fragmentation of a cfDNA sample using reads of cfDNA fragments is by identifying contexts of sites where cfDNA fragments separated from a DNA strand (referred to as “fragmentation sites”). Each cfDNA fragment has two fragmentation sites where it separated from a DNA strand. A fragmentation site context refers to a sequence of nucleotides that appear in the DNA strand at locations before and/or after a fragmentation site. The sequence of nucleotides may be obtained from a reference genome with which a read is aligned and/or from the read itself. For example, a fragmentation site context may be a nucleotide subsequence of a reference genome that spans a fragmentation site. As another example, a fragmentation site context may be a nucleotide subsequence in a read that occurs before or after the fragmentation site. Fragmentation site contexts of a cfDNA sample may be used to determine whether a cfDNA sample is cancerous and, if so, a tissue of origin of the cancer.

Described herein are improved techniques of performing cancer detection by analyzing fragmentation site contexts of a cfDNA sample. The inventors recognized that distributions of fragmentation site contexts (“fragmentation site context distributions”) of cancerous cfDNA samples deviate from fragmentation site context distributions of non-cancerous cfDNA samples. Accordingly, the inventors developed techniques of efficiently encoding a representation of a cfDNA sample's fragmentation site context distribution in one or more data structures that can be stored in a system's memory. The system uses the information stored in the data structure(s) to determine whether the cfDNA sample is cancerous. For example, the system may be configured to compare the data structure(s) to data structures encoding representations of fragmentation site context distributions of healthy and/or cancerous cfDNA samples. As another example, the system may be configured to process the data structure(s) using a machine learning (ML) model to obtain output indicating whether the cfDNA sample is cancerous. The system may be further configured to use the data structure(s) encoding a representation of a cfDNA sample's fragmentation site context distribution to determine a tissue of origin of cancerous cells in the cfDNA sample.

The techniques described herein for detecting the presence of cancer improve upon conventional techniques for detecting the presence of cancer. In particular, the techniques for detecting presence of cancer describe herein improves upon conventional techniques that use fragmentation site contexts to detect cancer. Embodiments of the system described herein reduce the amount of DNA sequencing data needed to perform cancer detection relative to conventional techniques. Specifically, embodiments of the system described herein require a lower number of cfDNA fragment reads to encode a representation of the cfDNA sample's fragmentation site context than the conventional techniques.

Conventional techniques determine a probability mass function (PMF) as a representation of a cfDNA sample's fragmentation site contexts. The PMF indicates probabilities for every possible nucleotide sequence that could occur as a fragmentation site context. For example, the PMF may be encoded in a matrix that includes a value for every possible nucleotide sequence that could form a fragmentation site context (e.g., 256 values for a context of 4 nucleotides, 1024 values for a context of 5 nucleotides, and 4096 values for a context of 6 nucleotides). In contrast, embodiments of the system described herein determine probabilities of nucleotide subsequences that could appear within a fragmentation site context, and encode those probabilities in a data structure representing the cfDNA sample's fragmentation site contexts. These nucleotide subsequences have a shorter length than the entire context, and thus have a fewer number of parameters for which to determine a probability of occurrence. Because the system needs to determine probabilities for a fewer number of possible parameters than conventional techniques, the system needs less sequencing data (i.e., fewer reads of cfDNA fragments) to determine the probabilities than conventional techniques. In the case of a context of 6 nucleotides, conventional techniques would require 256 more reads than embodiments of the technology described herein. In the case of a context of 4 nucleotides, conventional techniques would require 16 times more reads than embodiments of the technology described herein.

As an illustrative example demonstrating how techniques described herein use less DNA sequencing data than conventional techniques, for fragmentation site contexts of 6 nucleotides (referred to as “hexamers”) a PMF indicates probabilities for 46 (i.e., 4096) possible nucleotide sequences that could form the context. Generating a PMF representing a cfDNA sample's fragmentation site contexts requires estimating probabilities of all the possible nucleotide sequences, which is a total of 4096 parameters. In contrast, some embodiments described herein may represent a cfDNA sample's fragmentation site contexts as probabilities of dinucleotides subsequences that could appear at 5 different positions in a context. The system thus needs to estimate probabilities of 42 (i.e., 16) possible dinucleotides occurring at different positions. Because the system needs to estimate probabilities of a fewer number of parameters than is required for generating a PMF (i.e., 16 parameters vs. 4096 parameters), the system needs less DNA sequencing data than conventional techniques to achieve the same degree of precision.

By reducing the amount of DNA sequencing data (i.e., the number of reads) needed to perform cancer detection, techniques described herein reduce the depth of DNA sequencing that needs to be performed on cfDNA samples relative to conventional techniques. In other words, the techniques allow for shallower sequencing of cfDNA samples while providing similar or even improved cancer detection performance over existing techniques. By reducing the depth of sequencing that needs to be performed on cfDNA samples, the techniques increase the throughput of cfDNA samples that can be analyzed in a given time period. This provides a more efficient cancer detection system.

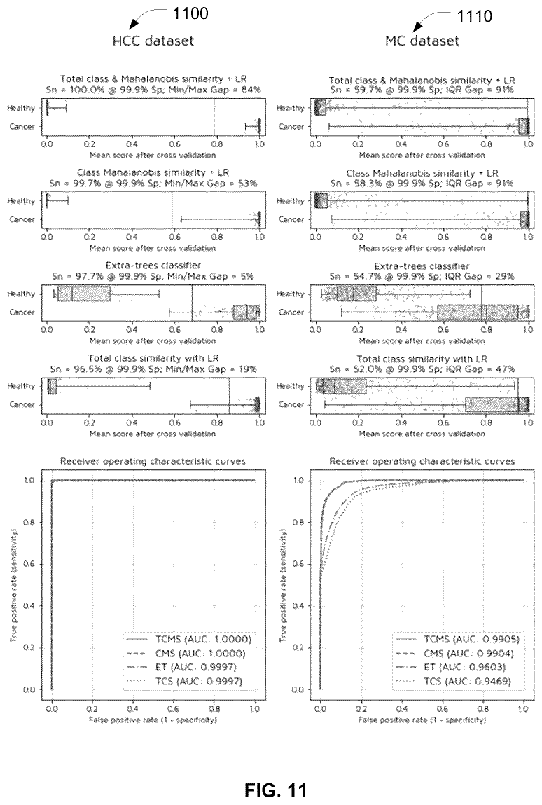

Moreover, techniques described herein provide improved techniques of determining whether a subject has cancer by analyzing fragmentation of a cfDNA sample from the subject. For example, the techniques more accurately classify cfDNA samples as cancerous or noncancerous than conventional techniques. The techniques were tested on datasets and show improved performance of determining whether a subject has cancer relative to conventional techniques, as discussed in more detail herein with reference to FIG. 11-FIG. 14.

Some embodiments provide a system for determining whether a subject has cancer by analyzing fragmentation in a cfDNA sample obtained from the subject. The system accesses sequencing data obtained from sequencing the cfDNA sample. The sequencing data includes reads of cfDNA fragments. The system aligns the reads to a reference (e.g., a reference human genome) and uses the results of the aligning to: (1) identify fragmentation sites of the cfDNA sample; and (2) fragmentation site contexts (e.g., nucleotide subsequences of the reference spanning the fragmentation sites, or nucleotide subsequences of the reads succeeding and preceding the fragmentation sites). The system uses the fragmentation site contexts (e.g., a hexamer spanning the fragmentation site) to generate a data structure (e.g., a position probability matrix (PPM)) encoding information about a fragmentation site context distribution of the cfDNA sample. The system uses the data structure to determine whether the subject has cancer. For example, the system may process the data structure using a machine learning model to obtain a classification of whether the cfDNA sample is cancerous. As another example, the system may compare the data structure to data structures associated with non-cancerous and/or cancerous cfDNA samples to obtain a classification of whether the cfDNA sample is cancerous. In some embodiments, the system may be configured to use the data structure to determine a tissue of origin of cancer cells.

In some embodiments, the data structure encoding information about the distribution of fragmentation site contexts of the cfDNA comprises g a data structure indicating, for each of a plurality of nucleotide sequences of a fixed length, estimated probabilities of the nucleotide sequence occurring at a plurality of fragmentation site context positions. For example, the data structure may be a PPM that indicates, for each of the plurality of nucleotide sequences of the fixed length, estimated probabilities of the nucleotide sequence occurring at the plurality of fragmentation site context positions. In some embodiments, the plurality of nucleotides of the fixed length are dinucleotides.

In some embodiments, determining the fragmentation site contexts of the cfDNA sample using the nucleotide subsequences of the reference corresponding to the fragmentation sites includes: determining a first one of the nucleotide subsequences corresponding to a first fragmentation site to be a first fragmentation site context, and determining a reverse complement of a second one of the nucleotide subsequences corresponding to a second fragmentation site as a second fragmentation site context of the fragmentation site contexts.

In some embodiments, determining whether the subject has cancer using the data structure encoding information about the distribution of fragmentation site contexts of the cfDNA sample includes: determining a measure of similarity (e.g., a measure of distance such as Jensen-Shannon distance (JSD) or Mahalanobis distance, or a measure of similarity derived therefrom) between the data structure (e.g., a PPM) and a first plurality of data structures (e.g., a first set of PPMs) encoding information about fragmentation site context distributions of cancerous cfDNA samples to obtain a first similarity measurement, and determining whether the subject has cancer using the first similarity measurement. In some embodiments, the system may be configured to determine the measure of similarity between the data structure and a second plurality of data structures (e.g., a second set of PPMs) encoding information about fragmentation site context distributions of non-cancerous cfDNA samples to obtain a second similarity measurement, and determine whether the subject has cancer using the first similarity measurement and/or the second similarity measurement.

In some embodiments, the system may be configured to project the data structure (e.g., the PPM) into a projection space to obtain a projection of the data structure, and determine whether the subject has cancer using the projection of the data structure. The system may be configured to determine whether the subject has cancer using projections into the projection space of: a first plurality of data structures (e.g., a first set of PPMs) encoding information about fragmentation site context distributions of cancerous cfDNA samples, and/or a second plurality of data structures (e.g., a second set of PPMs) encoding information about fragmentation site context distributions of non-cancerous cfDNA samples.

In some embodiments, the system may be configured to determine the cancer's tissue of origin using the data structure encoding information about the fragmentation site context distribution of the cfDNA sample by determining similarity measurements between the data structure (e.g., a PPM) encoding information about the distribution of fragmentation site contexts of the cfDNA sample and a plurality of reference data structure sets (e.g., sets of PPMs) each associated with a tissue of origin and including data structures encoding information about distributions of fragmentation site contexts of cfDNA samples with cancer from the tissue of origin.

In some embodiments, the system may be configured to determine whether the subject has a particular one of multiple cancer types using the data structure encoding information about the distribution of fragmentation site contexts of the cfDNA sample. The system may be configured to determine a measure of similarity (e.g., a measure of distance or a measure of similarity derived therefrom) between the data structure and multiple cancer-specific sets of data structures to obtain multiple similarity measurements for the multiple cancer types. The multiple cancer-specific sets of data structures may each encode information about fragmentation site context distributions of cancerous cfDNA samples of a respective one of the multiple cancer types. The system may be configured to determine whether the subject has a particular one of the multiple cancer types using the multiple similarity measurements for the multiple cancer-specific sets of data structures.

In some embodiments, the system may be configured to determine an intervention for the subject based on the cancer's tissue of origin. In some embodiments, the system may be configured to trigger administration of treatment to the patient when it is determined that the cfDNA sample is cancerous.

Following below are more detailed descriptions of various concepts related to, and embodiments of, cancer detection systems and methods developed by the inventors. It should be appreciated that various aspects described herein may be implemented in any of numerous ways. Examples of specific implementations are provided herein for illustrative purposes only. In addition, the various aspects described in the embodiments below may be used alone or in any combination and are not limited to the combinations explicitly described herein.

The term “cancer”, as may be used herein, refers to a cell or population of cells characterized by uncontrolled proliferation. The term “tumor”, as may be used herein, refers to a contiguous population of cells. A tumor may be benign, meaning that it is localized to a single tissue, or malignant, meaning that it is cancerous and capable of spreading to other parts of the body through the circulatory and/or lymphatic system. Cells may become cancerous as a result of accumulated mutations in their genome. Examples of cancer include but are not limited to colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

The terms “cell-free DNA” or “cfDNA”, as may be used interchangeably herein, refer to deoxyribonucleic acid species that occur extracellularly. Cell-free DNA may originate from one or more cells. Cell-free DNA may originate from one or more cell types. Cell-free DNA may originate from healthy cells or diseased cells. Cell-free DNA may be single-stranded or double stranded. Cell-free DNA originates from the cells of a subject. Cell-free DNA may originate from both healthy and diseased cells of a subject. Cell-free DNA encodes one or more genes belonging to the subject's genome. Cell-free DNA may contain mutations that are indicative of a disease, such as a cancer.

The term “biological sample” as used herein generally refers to a tissue or body fluid sample derived from a subject. Biological samples can be obtained directly from a subject. The biological sample can be or can comprise one or more nucleic acid molecules, such as deoxyribonucleic acid (DNA) or ribonucleic acid (RNA) molecules (e.g., cfDNA). The biological sample may be derived from any organ, tissue or biological fluid. The biological sample may comprise, for example, body fluids or solid tissue samples. An example of a solid tissue sample is a tumor sample, e.g., a solid tumor biopsy. Body fluids include, for example, blood, serum, plasma, tumor cells, saliva, urine, lymphatic fluid, synovial fluid, interstitial fluid, cerebrospinal fluid, prostate fluid, semen, sputum, mucus, gastric acid, bile, feces, tears, and derivatives thereof. A cfDNA sample may be extracted from a biological sample.

As used herein, “subject” means a human or animal. Usually, the animal is a vertebrate such as a primate, rodent, livestock animal, or hunting animal. Primates include chimpanzees, cynomolgus monkeys, spider monkeys, and macaques such as rhesus monkeys. Rodents include mice, rats, hamsters, rabbits, guinea pigs, squirrels, woodchucks, ferrets. Livestock and game animals include cattle, horses, pigs, deer, bison, buffalo, cat species such as domesticated cats, dog species such as domesticated dogs, foxes, wolves, birds such as chickens, turkeys, ducks, geese, emus, ostriches, and fish such as trout, catfish, and salmon. In some embodiments, the subject is a mammal, such as a primate, such as a human. The terms “individual”, “patient” and “subject” are used interchangeably herein. Preferably, the subject is a mammal. The mammal can be a human, non-human primate, mouse, rat, dog, cat, horse, or cow, but is not limited to these examples. Mammals other than humans can be conveniently used, for example, as subjects that represent animal models of cancer, e.g., a particular type of cancer, such as, lung cancer. The subject can be male or female.

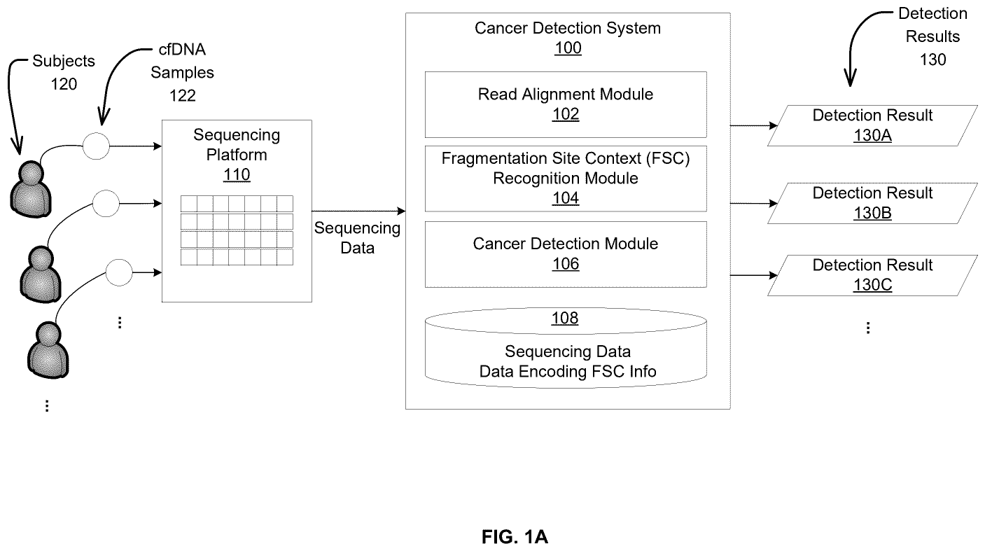

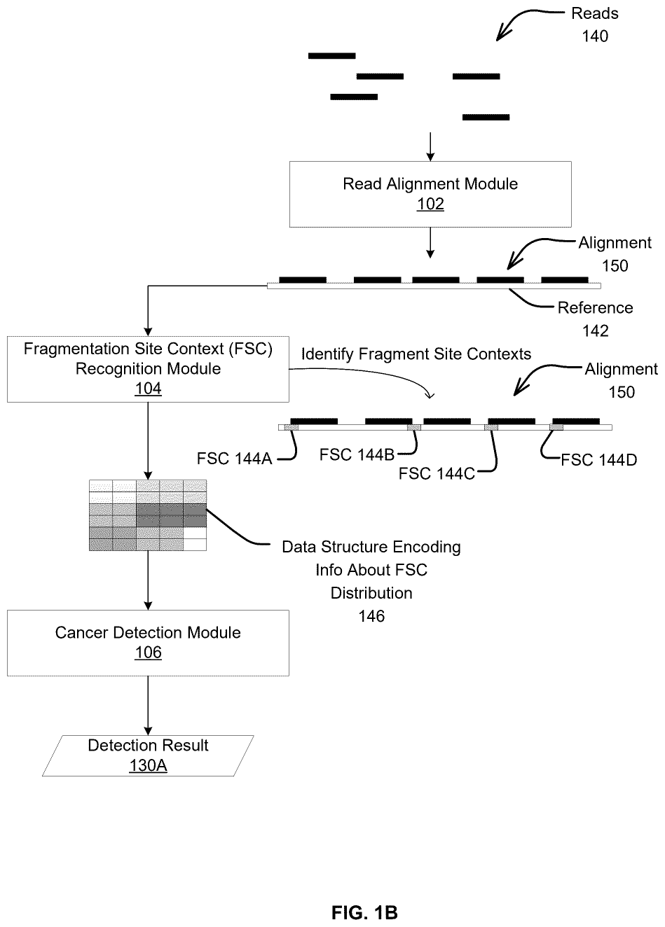

FIG. 1A illustrates an example computing environment in which some embodiments of the technology described herein may operate. The computing environment 100 includes multiple computing assets 106A, 106B, 106C. The environment includes a sequencing platform 110 for sequencing cfDNA samples 122 obtained from subjects 120. The environment further includes a cancer detection system 100 that accesses sequencing data generated by the sequencing platform 102. The cancer detection system 100 uses the sequencing data to determine the detection results 130A, 130B, 130C which indicate whether the subjects 120 have cancer. In some embodiments, if a subject is determined to have cancer a detection result may also indicate a tissue of origin of the cancer.

As illustrated in FIG. 1A, the cfDNA samples 122 are obtained from respective ones of the subjects 120. In some embodiments, the cfDNA samples 122 may be extracted from biological samples obtained from the subjects 120. For example, the biological samples may be blood samples (e.g., serum or plasma) obtained from the subjects 120. A cfDNA sample may be obtained from a particular subject by drawing blood into a tube and then extracting the cfDNA sample from the blood. The tube may be transported to another site for sequencing by the sequencing platform 110. In some embodiments, the cfDNA samples 122 may be extracted from other biological samples of the subjects 120 in addition to or instead of blood samples. For example, the cfDNA samples 122 may be extracted from saliva samples, urine samples, cerebrospinal fluid samples, and/or pleural fluid samples. Techniques described herein are not limited to a particular type of biological sample from which the cfDNA samples 122 are obtained.

The cfDNA samples 122 may be extracted from biological samples obtained from the subjects 120 using any suitable technique. For example, cfDNA samples 122 may be extracted from blood samples obtained from the subjects 120 by isolating plasma from the blood samples, and extracting cfDNA from the plasma. The cfDNA samples 122 may further be prepared for sequencing by the sequencing platform 110. For example, the cfDNA samples 122 may be prepared using a cfDNA sample preparation kit.

As illustrated in FIG. 1A, the sequencing platform 110 performs DNA sequencing on the cfDNA samples 122 to generate sequencing data comprising of reads of cfDNA fragments. In some embodiments, the sequencing platform 110 may be configured to perform whole genome sequencing (WGS) of a cfDNA sample. In some embodiments, the sequencing platform 110 may be configured to perform shallow WGS (sWGS) (also referred to as low-pass WGS). sWGS may be WGS with a sequencing coverage of less than 1×. In some embodiments, the sequencing platform 110 may be configured to perform deep WGS. Deep WGS may have a sequencing coverage of 30× or more. In some embodiments, the sequencing platform 110 may be configured to perform short-read DNA sequencing which generates reads of 300 or less base pairs. In some embodiments, the sequencing platform 110 may be configured to perform long-read DNA sequencing which generates reads of greater than 300 base pairs.

In some embodiments, the sequencing platform 110 may be any suitable sequencing platform. For example, the sequencing platform 110 may be a next-generation sequencing (NGS) platform. As another example, the sequencing platform 110 may be a third-generation sequencing platform. As another example, the sequencing platform 110 may be configured to perform Sanger sequencing. The sequencing platform 110 may be configured to perform sequencing to generate the sequencing data (e.g., for use by the cancer detection system 100). In some embodiments, the sequencing platform 110 may be a benchtop sequencing platform. In some embodiments, the sequencing platform 110 may be a production scale sequencing platform. Example sequencing platforms that may be used to sequence the cfDNA samples 122 include the MiSeq Benchtop Sequencer, the PacBio Sequel II, the HiSeq X Ten, the Oxford Nanopore PromethION, the NextSeq 500 Sequencer, NovaSeq6000, or the Oxford Nanopore MinION. Embodiments described herein may employ any suitable sequencing platform.

In some embodiments, the sequencing platform 110 may be configured to sequence a cfDNA sample by breaking the cfDNA sample into cfDNA fragments and sequencing the cfDNA fragments to obtain reads. Each of the reads may be a sequence of nucleotides of a respective cfDNA fragment. In some embodiments, the reads may be of different lengths. In some embodiments, each of the cfDNA samples 122 may have an associated set of sequencing data (e.g., a set of reads) generated by the sequencing platform 110 by sequencing the cfDNA sample. In some embodiments, the sequencing platform 110 may be configured to sequence the cfDNA fragments to obtain the reads by performing any suitable sequencing. For example, the sequencing platform 110 may be configured to perform Sanger sequencing. As another example, the sequencing platform 110 may be configured to perform next-generation sequencing (NGS) to obtain the reads.

As illustrated in FIG. 1A, the cancer detection system 100 may be configured to access the sequencing data generated by the sequencing platform 110 and store the sequencing data in data storage 108 of the cancer detection system 100. In some embodiments, the cancer detection system 100 may be configured to access the sequencing data through a communication network (e.g., through the Internet). For example, the cancer detection system 100 may receive the sequencing data from a computing device of the sequencing platform 110. As another example, the sequencing data generated by the sequencing platform 110 may be stored in a database and the cancer detection system 100 may be configured to access the sequencing data by querying the database. In some embodiments, the cancer detection system 100 may be implemented on a computing device integrated with the sequencing platform 110. The sequencing data generated by the sequencing platform 110 may be stored by the sequencing platform 110 in the data storage 108 of the cancer detection system 100.

The cancer detection system 100 may be configured to use the sequencing data to perform cancer detection (e.g., to determine whether the subjects 120 have cancer and/or a tissue of origin of cancer for subjects that are determined to have cancer). The cancer detection system 100 includes multiple modules including a read alignment module 102, a fragmentation site context (FSC) recognition module 104 and a cancer detection module 106. The cancer detection system 100 further includes data storage 108 storing the sequencing data accessed by the cancer detection system 100 and data encoding information about FSCs of the cfDNA samples 122 (e.g., information about FSC distributions of the cfDNA samples 122).

The data storage 108 may be any suitable memory for storing sequencing data and data encoding information about fragmentation site contexts. In some embodiments, the data storage 108 of the cancer detection system 100 may comprise of storage hardware. For example, the data storage 108 may include one or more hard drives (e.g., solid state drive(s) (SSD(s)) and/or disk drive(s)). In some embodiments, the data storage 108 may include a database storing the sequencing data and the data encoding information about fragmentation site contexts (e.g., data structures encoding information about fragmentation site contexts). Although in the example of FIG. 1A the data storage 108 is shown as part of the cancer detection system 100, in some embodiments, the data storage 108 may be remote from a computing device configured to execute the modules 102, 104, 106. For example, the data storage 108 may be storage in a data center that is remote from the computing device.

As illustrated in FIG. 1A, in some embodiments, the cancer detection system 100 may be configured to output the detection results 130. For example, the cancer detection system 100 may be configured to transmit the detection results 130 to another system (e.g., through a communication network). As another example, the cancer detection system 100 may be configured to generate output in a graphical user interface (GUI) indicating the detection results 130 (e.g., indicating whether subjects 120 have cancer and, optionally, a cancer tissue of origin for subjects determined to have cancer). In some embodiments, the detection results 130 may be used for downstream processing (e.g., as part of an automated treatment pipeline). For example, the detection results 130 may be used by an electronic health record (EHR) system to trigger alerts (e.g., in a GUI) about any of the subjects 120 that are determined to have cancer. As another example, the EHR system may automatically populate clinical records associated with the subjects 120 based on the detection results 130 (e.g., by storing information indicating whether the subjects 120 have cancer and/or a tissue of origin of detected cancer).

The operation of the modules 102, 104, 106 of the cancer detection system 100 will be described herein with reference to FIG. 1B. FIG. 1B shows the interaction between the modules 102, 104, 106 of the cancer detection system 102, according to some embodiments of the technology described herein.

As shown in FIG. 1B, the read alignment module 102 aligns the reads 140 of cfDNA fragments (e.g., obtained from sequencing one of the cfDNA samples 122) with a reference 142 to obtain the alignment 150. In some embodiments, the reference may be a human reference genome. For example, the human reference genome may be the GRCh38.p14 genome developed by the Genome Reference Consortium (GRC), the T2T-CHM13 genome developed by the Telomere-to-Telomere (T2T) Consortium, or another suitable reference genome. The read alignment module 102 may be configured to generate the alignment 150 of reads 140 with the reference 142 shown in FIG. 1B. The alignment 150 may indicate a mapping of each of the reads 140 to a respective portion of the reference 142. In the alignment 150, the nucleotides of each read may be aligned with a subsequence of nucleotides in the reference 142.

In some embodiments, the read alignment module 102 may be configured to align the reads 140 with the reference 142 using an alignment algorithm. The read alignment module 102 may be configured to apply the alignment algorithm to the reads 140 and the reference 142 to obtain the alignment 150. For example, the read alignment module 102 may use the BWA-MEM2 alignment algorithm described in Vasimuddin, Md. et al. “Efficient Architecture-Aware Acceleration of BWA-MEM for Multicore Systems.” 2019 IEEE International Parallel and Distributed Processing Symposium (IPDPS) (2019): 314-324. As another example, the read alignment module 102 may use the Bowtie 2 alignment algorithm described in Langmead B, Salzberg SL “Fast gapped-read alignment with Bowtie 2.” Nat Methods. 2012 Mar. 4; 9(4):357-9. The alignment algorithms mentioned herein are example alignment algorithms that may be used by the read alignment module 102. In some embodiments, the read alignment module 102 may be configured to use any suitable alignment algorithm.

In some embodiments, the read alignment module 102 may be configured to filter the reads 140 to obtain a filtered set of reads that the read alignment module 102 aligns with the reference 142 to obtain the alignment 150. In some embodiments, the read alignment module 102 may be configured to filter out reads containing adapters. In some embodiments, the read alignment module 102 may be configured to filter the reads 140 by: (1) determining scores for the reads 140 indicating quality of the reads 140; and (2) filtering the reads 140 using the scores (e.g., by filtering out reads that do not meet a minimum score threshold). The read alignment module 102 may determine the scores based on length of the reads 140, a percentage of read bases that were not determined, and/or other criteria.

In some embodiments, the read alignment module 102 may be configured to filter out reads from an initial alignment to obtain the alignment 150. The read alignment module 102 may be configured to: (1) apply an alignment algorithm to a set of reads to obtain the initial alignment; and (2) and filter the set of reads based on the initial alignment. For example, the read alignment module 102 may filter out non-primary alignments (e.g., secondary or supplementary alignments), improperly paired reads, polymerase chain reaction (PCR) duplicate reads, optical duplicate reads, and/or reads that fail a quality check. In some embodiments, the read alignment module 102 may be configured to determine scores for the reads in the initial alignment (e.g., based on a percentage of the reads that match corresponding portions of the reference 142). The read alignment module 102 may be configured to use the scores to filter out reads from the initial alignment (e.g., by removing reads from the initial alignment that do not meet or exceed a threshold score).

As shown in FIG. 1B, the FSC recognition module 104 may be configured to identify FSCs using the alignment 150. In the example of FIG. 1B, the FSC recognition module 104 identifies FSC 144A, FSC 144B, FSC 144C, FSC 144D. In some embodiments, the FSC recognition module 104 may be configured to identify the FSCs 144A, 144B, 144C, 144D by: (1) identifying fragmentation sites in the reference 142 using the alignment 150; and (2) identifying FSCs corresponding to the fragmentation sites. The FSC recognition module 104 may be configured to identify the fragmentation sites by identifying points in the reference 142 associated with where a read begins. The FSC recognition module 140 may be configured to identify fragmentation sites in the reference 142 using the alignment 150 by identifying points of the reference that lie between a portion of the reference aligned with a read (i.e., internal to a cfDNA fragment) and a portion of the reference that is not aligned with the read (i.e., external to the cfDNA fragment). In some embodiments, the FSC recognition module 104 may be configured to indicate a fragmentation site as a coordinate indicating a point in the reference 142. For example, the FSC recognition module 104 may determine the coordinate as a nucleotide position in the reference 142.

Each cfDNA fragment in a cfDNA sample may have two fragmentation sites. In some embodiments, the FSC recognition module 104 may be configured to identify both fragmentation sites associated with each cfDNA fragment. The alignment 150 may include mated pairs of reads where one read of a pair corresponds to a first portion of a cfDNA fragment and a second read in the pair corresponds to a second portion of the cfDNA fragment. The FSC recognition module 104 may be configured to identify the two fragmentation sites associated with the cfDNA fragment by identifying a point in the reference 142 corresponding to a beginning of the first read as a first fragmentation site and a point in the reference 142 corresponding to a beginning of the second read as a second fragmentation site.

The FSC recognition module 104 may be configured to identify an FSC corresponding to a fragmentation site of a cfDNA fragment by identifying a nucleotide sequence associated with the fragmentation site. In some embodiments, the FSC recognition module 104 may be configured to identify the FSC using a subsequence of the reference 142 corresponding to the fragmentation site. For example, the FSC recognition module 104 may be configured to: (1) identify a nucleotide subsequence of the reference 142 that spans the fragmentation site; and (2) select the identified nucleotide subsequence as the FSC corresponding to the fragmentation site. As another example, the FSC recognition module 104 may be configured to: (1) identify a nucleotide subsequence of the reference 142 that spans the fragmentation site; and (2) determine a reverse complement of the nucleotide subsequence as the FSC corresponding to the fragmentation site (e.g., because the read may be aligned to a reverse strand).

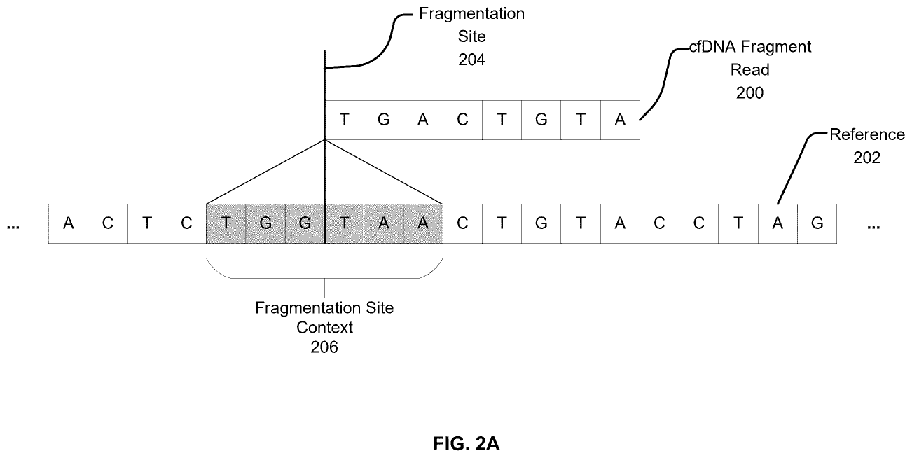

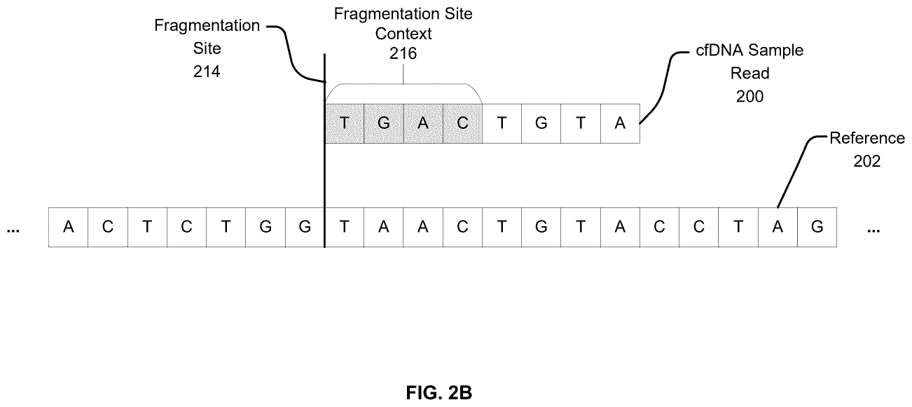

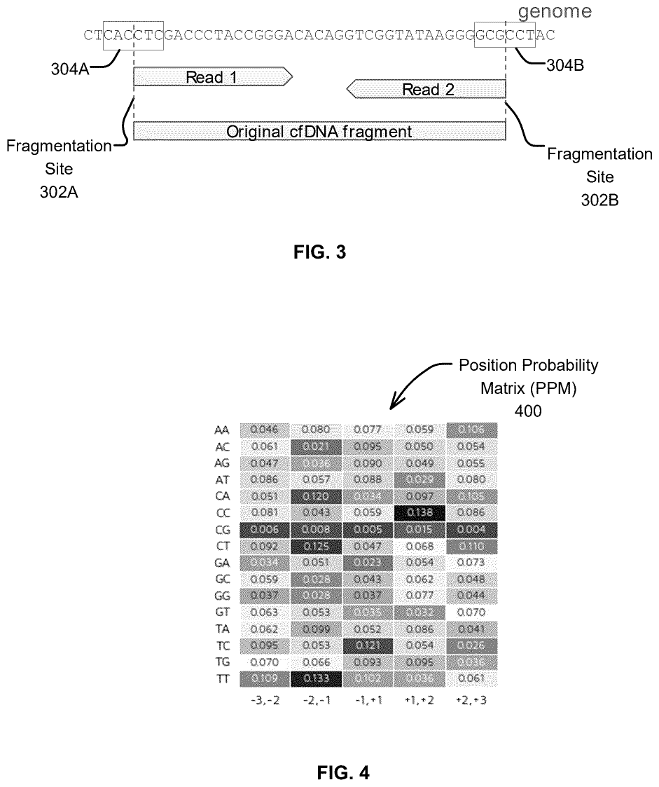

In some embodiments, a subsequence identified in the reference 142 may include one or more nucleotides that precede the fragmentation site and one or more nucleotides that follow the fragmentation site. The subsequence may consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or other suitable number of nucleotides. For example, the FSC recognition module 104 may identify a hexamer spanning the fragmentation site, where the hexamer consists of the three nucleotides in the reference 142 that precede the fragmentation site and the three nucleotides in the reference 142 that follow the fragmentation site. As another example, the FSC recognition module 104 may identify a subsequence of four nucleotides spanning the fragmentation site that consists of two nucleotides in the reference 142 that precede the fragmentation site and two nucleotides in the reference 142 that follow the fragmentation site. An example of identifying a subsequence of the reference 142 from which to determine an FSC is described herein with reference to FIG. 2A and FIG. 3.

In some embodiments, the FSC recognition module 104 may be configured to identify, as an FSC corresponding to a fragmentation site of a cfDNA fragment, a subsequence of a read of the cfDNA fragment as the FSC corresponding to the fragmentation site. The subsequence may be a portion of the read that follows the fragmentation site or a portion of the reference that precedes the fragmentation site. The subsequence of the read may consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or other suitable number of nucleotides. For example, the subsequence may consist of 4 nucleotides that precede or follow a fragmentation site.

Example FSC lengths described herein are for illustrative purposes. In some embodiments, the length of the FSC identified by the FSC recognition module 104 may be configurable. For example, the FSC recognition module 104 may store a configurable parameter that indicates the length of FSCs to be identified. The parameter may be configured based on configuration settings (e.g., set by a file and/or user input received through a graphical user interface (GUI)). The FSC recognition module 104 may configure its identification of FSCs based on the value of the parameter.

Example numbers of nucleotides in an FSC on each side of a corresponding fragmentation site are for illustrative purposes. Different numbers of nucleotides on each side of the fragmentation site may be used than in the examples described herein. In some embodiments, the number of nucleotides on each side of the fragmentation site to include in an FSC may be a configurable parameter (e.g., set by a file and/or user input received through a GUI). The FSC recognition module 104 may configure its identification of FSCs based on the value of the parameter.

The FSC recognition module 104 generates a representation of the FSCs identified using the alignment 150. As illustrated in FIG. 1B, in some embodiments, the FSC recognition module 104 may be configured to generate a data structure 146 that encodes information about an FSC distribution of the cfDNA sample. The data structure 146 may indicate a fragmentation pattern of the cfDNA sample. In some embodiments, the data structure may be any suitable data structure. For example, the data structure may be an array, vector, matrix, linked list, a graph, tree, or any other suitable data structure.

For example, in some embodiments, the data structure 146 may be a position probability matrix (PPM) in which each element indicates a probability of a particular nucleotide sequence of fixed length (e.g., a dinucleotide) occurring at an FSC position. For example, each element in the PPM may indicate the probability of a particular dinucleotide occurring at an FSC position. The number of nucleotide sequences of the fixed length for which a probability is indicated may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or another suitable number of nucleotide sequences. For example, the PPM may indicate for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or another suitable number of dinucleotides. To illustrate, the rows of the PPM may each represent a particular dinucleotide and the columns of the PPM may each represent an FSC position. Accordingly, the PPM would have 16 rows (i.e., one for each dinucleotide). The number of columns would be equivalent to one less than the number of nucleotides in an FSC (e.g., 5). An example of such a PPM is described herein with reference to FIG. 4. As another example, each clement in the PPM may indicate the probability of a particular nucleotide occurring at an FSC position. To illustrate, the rows of the PPM may each represent a particular nucleotide and the columns of the PPM may each represent an FSC position. Accordingly, the PPM would have 4 rows (i.e., one for each nucleotide). The number of columns would be equivalent to the number of nucleotides in an FSC (e.g., 6).

In some embodiments, the FSC recognition module 104 may be configured to determine probabilities indicated by a PPM. The FSC recognition module 104 may be configured to determine the probabilities using the FSCs identified using the alignment 150. The FSC recognition module 104 may be configured to determine the probabilities using the FSCs by: (1) determining a number of occurrences of each nucleotide sequence (e.g., dinucleotide) at each of FSC positions; and (2) dividing the number of occurrences by the total number of FSCs to obtain the probabilities indicated by the PPM.

It should be appreciated that a PPM data structure provides a representation of the FSCs of a cfDNA sample that stores less information than a data structure indicating a PMF. The PPM has far fewer rows than a PMF matrix. To illustrate, for FSCs with a length of 6 nucleotides, a PMF matrix would have 4⁶ rows (i.e., 4096 rows), where each row indicates a probability of a particular combination of 6 nucleotides that may occur in an FSC. By contrast, a PPM that indicates probabilities of dinucleotides has only 4² rows (i.e., 16 rows) and 5 columns representing the FSC positions. The PPM would thus have probabilities of 16 different dinucleotides. As the PPM stores less information, it requires fewer reads of cfDNA fragments to obtain the information. The PPM thus allows for the use of shallower sequencing by the sequencing platform 110 and use of lower cost assays for sequencing.

As illustrated in FIG. 1B, the cancer detection module 106 uses the data structure 146 (e.g., a PPM) to determine a cancer detection result 130A for a cfDNA sample. In some embodiments, the cancer detection module 106 may be configured to use the data structure 146 to determine whether the cfDNA sample is cancerous. For example, the detection result 130A may include a classification of whether the cfDNA sample is cancerous. In some embodiments, the cancer detection module 106 may be configured to use the data structure 146 to determine a tissue of origin of cancerous cells when the cfDNA sample is determined to be cancerous. For example, the detection result 130A may include a classification of tissue of origin (e.g., bile duct cancer, breast cancer, colorectal cancer, gastric cancer, lung cancer, ovarian cancer, pancreatic cancer, or another type of cancer).

In some embodiments, the cancer detection module 106 may be configured to determine whether the cfDNA sample is cancerous using the data structure 146 by comparing the data structure 146 to a reference set of data structures associated with known healthy cfDNA samples and/or to a reference set of data structures associated with known cancerous cfDNA samples. A reference set of data structures (e.g., PPMs) may be generated using a labeled set of FSCs. For example, a reference set of data structures representing FSC distributions of non-cancerous cfDNA samples may be generated using FSCs labeled as non-cancerous (e.g., from an existing database). As another example, a reference set of data structures representing FSC distributions of cancerous cfDNA samples may be generated using FSCs labeled as cancerous (e.g., from an existing database). The cancer detection module 106 may be configured to determine a similarity measurement between the data structure 146 and a reference set of data structures. The cancer detection module 106 may be configured to use the similarity measurement to classify the cfDNA sample as being cancerous or not.

In some embodiments, the cancer detection module 106 may be configured to use a similarity measurement between the data structure 146 and a reference set of data structures to determine whether the cfDNA sample is cancerous. The cancer detection module 106 may be configured to determine a classification of the cfDNA sample to be output in the detection result 130A. In some embodiments, the cancer detection module 106 may be configured to: (1) determine a first similarity measurement between the data structure 146 and a first reference set of data structures representing non-cancerous cfDNA samples (also referred to as “healthy cfDNA samples”); (2) determine a second similarity measurement between the data structure 146 and a second reference set of data structures representing cancerous cfDNA samples; and (3) determine a classification of whether the cfDNA sample is cancerous using the first and second similarity measurements. For example, the cancer detection module 106 may classify the cfDNA sample as cancerous when the second similarity measurement is greater than the second similarity measurement and as non-cancerous (or healthy) when the second similarity is less than the first similarity. As another example, the cancer detection module 106 may classify the cfDNA sample as cancerous when the second similarity is a threshold amount greater than the first similarity measurement. In some embodiments, the threshold amount may be a value in one of the ranges 0-0.1, 0.1-0.2, 0.2-0.3, 0.3-0.4, or 0.4-0.5. For example, the threshold amount may be 0.2.

In some embodiments, the cancer detection module 106 may be configured to determine the similarity measurement by: (1) transforming the data structure 146; and (2) comparing the transformation of the data structure 146 to transformations of the reference set of data structures. For example, the cancer detection module 106 may transform a PPM into a vector of numerical values and determine a similarity measurement between the normalized vector and vectors representing a reference set of data structures.

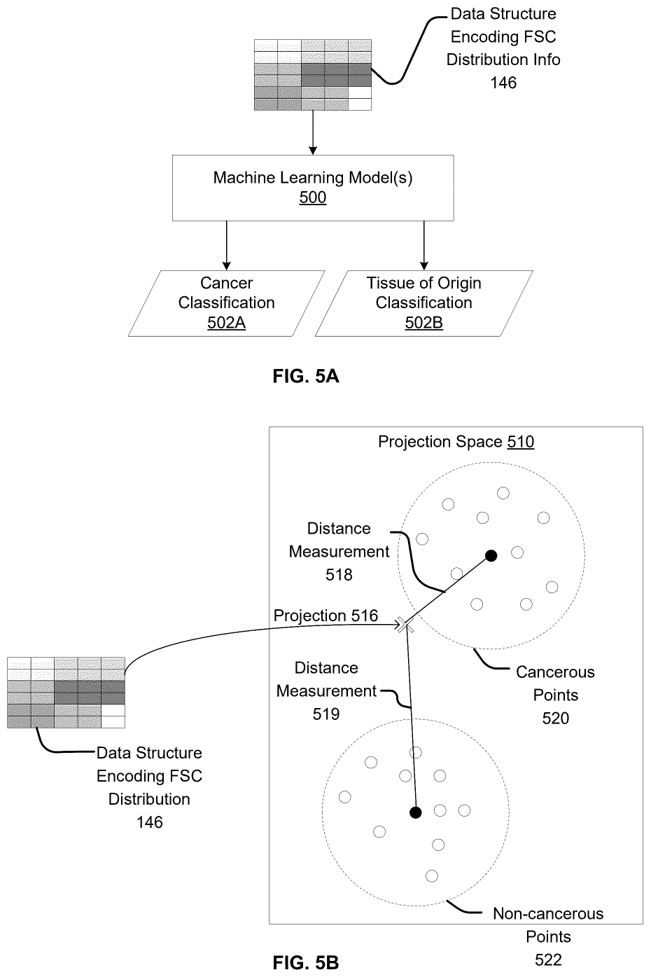

In some embodiments, the cancer detection module 106 may be configured to determine similarity measurements using a measure of similarity that is based on a measure of distance between the data structure 146 and the reference set of data structures. For example, the cancer detection module 106 may determine a distance measurement between the data structure 146 and the reference set of data structures and determine a similarity measurement based using the distance measurement. Example measures of distance that may be used are described herein with reference to FIG. 5B. In some embodiments, the measure of similarity may be the measure of distance. In some embodiments, the measure of similarity may be derived from the measure of distance. This may allow the summation of similarities to estimate the similarity of the data structure 146 to a set of multiple data structures. Examples of how a measure of similarity may be derived from a measure of distance are described herein with reference to FIG. 5B.

In some embodiments, the cancer detection module 106 may be configured to determine a classification of whether the cfDNA sample is cancerous using a distance-based classifier. For example, the cancer detection module 106 may determine a classification by determining a distance to a centroid of data structures representing healthy cfDNA samples, a distance to k nearest neighbors, or a local outlier factor. As another example, the cancer detection module 106 may use radius neighbors classifiers. In some embodiments, the cancer detection module 106 may be configured to determine a classification of whether the cfDNA sample is cancerous using anomaly detection based on reconstruction error. The cancer detection module 106 may determine a reconstruction error for the data structure 146 by: (1) projecting the data structure 146 or a derivative thereof into a space to obtain a projection; (2) generating a reconstruction using the projection; and (2) comparing the reconstruction to the original data structure 146 or derivative thereof to obtain a reconstruction error. For example, the cancer detection module 106 may determine the reconstruction error using a reconstruction obtained from an autoencoder, nonnegative matrix factorization, or principle component analysis (PCA).

In some embodiments, the cancer detection module 106 may be configured to determine a classification of whether the cfDNA sample is cancerous using a machine learning model. The cancer detection module 106 may be configured to use the data structure 146 (e.g., a PPM) and/or a derivative thereof as a set of features. The cancer detection module 106 may be configured to provide the set of features as input to the machine learning model to obtain an output indicating the classification. In some embodiments, the cancer detection module 106 may be configured to use any suitable machine learning model. Example machine learning models are described herein with reference to FIG. 5A.

In some embodiments, the cancer detection module 106 may be configured to determine a tissue of origin of a cfDNA sample that is determined to be cancerous. The cancer detection module 106 may be configured to determine the tissue of origin by determining a classification of the data structure 146 into one of multiple classes representing respective tissues of origin.

In some embodiments, the cancer detection module 106 may be configured to determine the tissue of origin by using a machine learning model to determine a classification of the data structure 146 into one of multiple classes each representing a respective tissue of origin. The cancer detection module 106 may be configured to use the data structure 146 or a derivative thereof as a set of features. The cancer detection module 106 may be configured to provide the set of features as input to the machine learning model to obtain a corresponding output indicating a classification. Example machine learning models and training techniques that may be used to determine the classification are described herein.

In some embodiments, the cancer detection module 106 may be configured to determine a classification of the data structure 146 into one of the classes using a similarity-based classification. The cancer detection module 106 may be configured to determine similarity measurements between the data structure 146 and reference sets of data structures representing respective tissues of origin. Example measures of similarity that may be used are described herein. The data structures in a reference set may be generated from FSCs of cfDNA samples with cancer known to originate from the tissue of origin represented by the reference set. The cancer detection module 106 may be configured to determine a tissue of origin classification using the similarity measurements. For example, the cancer detection module 106 may determine the classification by: (1) identifying, using the similarity measurements, the reference set of data structures that are most similar to the data structure 146; and (2) determining the classification to be the tissue of origin represented by the reference set of data structures.

FIG. 2A illustrates identification of an FSC 206 corresponding to a fragmentation site 204, according to some embodiments of the technology described herein. The identification of the FSC 206 illustrated in FIG. 2A may be performed by any suitable computing device. For example, the identification of the FSC 206 may be performed by the FSC recognition module 104 of cancer detection system 100 described herein with reference to FIG. 1A-FIG. 1B.

FIG. 2A shows a cfDNA fragment read 200 aligned with a reference 202 (e.g., a human reference genome). The alignment of the read 200 with the reference 202 may, for example, have been performed by the read alignment module 102 of the cancer detection system described herein with reference to FIG. 1A-FIG. 1B. In the example of FIG. 2A, the system identifies a subsequence of the reference 202 spanning the fragmentation site 204 as the FSC 206. Specifically, the system identifies a hexamer of the reference 202 spanning the fragmentation site 204 as the FSC 206. The identified FSC 206 is shaded and consists of the following subsequence of nucleotides in the reference 202: thymine (T), guanine (G), guanine (G), thymine (T), adenine (A), adenine (A). The hexamer consists of the three nucleotides in the reference 202 that precede the fragmentation site 204 and the three nucleotides in the reference 202 that follow the fragmentation site 204. Thus, a portion of the hexamer is external to the cfDNA fragment and a portion of the hexamer is internal to the cfDNA fragment. Examples of other types of FSCs that the system may be configured to identify are described herein with reference to the FSC recognition module 104.

As illustrated by the example of FIG. 2A, the read 200 may not completely match a portion of the reference 202 with which the read 200 is aligned. For example, the first three nucleotides in the read 200 are “TGA” whereas the subsequence of the reference 202 with which the read 200 is aligned begins with “TAA”. In the example embodiment of FIG. 2A, the system identifies a subsequence of the reference 202 as the FSC 206. In some embodiments (e.g., as described with reference to FIG. 2B), the system may be configured to identify a subsequence of the read 200 as the FSC.

FIG. 2B illustrates identification of an FSC 216 corresponding to a fragmentation site 214, according to some embodiments of the technology described herein. The identification of the FSC 216 illustrated in FIG. 2B may be performed by any suitable computing device. For example, the identification of the FSC 216 may be performed by the FSC recognition module 104 of cancer detection system 100 described herein with reference to FIG. 1A-FIG. 1B.

FIG. 2B shows the cfDNA fragment read 200 aligned with the reference 202 (e.g., a human reference genome). In the example of FIG. 2B, the system identifies a subsequence of the read 200 as the FSC 216. Specifically, the system identifies a subsequence of four nucleotides following the fragmentation site 214. The subsequence of nucleotides in the read 200 is “TGAC”. As the FSC is identified in the read, the entire FSC is internal to the cfDNA fragment indicated by the read 200. Examples of other types of FSCs that the system may be configured to identify are described herein with reference to the FSC recognition module 104. As illustrated by the example of FIG. 2B, the read 200 may not completely match a portion of the reference 202 with which the read 200 is aligned. For example, the first three nucleotides in the read 200 are “TGA” whereas the subsequence of the reference 202 with which the read 200 is aligned begins with “TAA”. In the example embodiment of FIG. 2B, the system identifies a subsequence of the read 200 as the FSC 216. In some embodiments (e.g., as described with reference to FIG. 2A), the system may be configured to identify a subsequence of the reference 202 as the FSC 216.

FIG. 3 illustrates an example of identifying FSCs corresponding to two fragmentation sites 302A, 302B of a cfDNA fragment, according to some embodiments of the technology described herein. The identification of the illustrated in FIG. 3 may be performed by any suitable computing device. For example, the identification of the FSCs may be performed by the FSC recognition module 104 of cancer detection system 100 described herein with reference to FIG. 1A-FIG. 1B.

In the example of FIG. 3, the system has identified subsequences 304A, 304B in a reference genome (e.g., a human reference genome) to use for determining FSCs. In the example of FIG. 3, the first read (labeled “Read 1”) is aligned to a forward strand while the second read (labeled “Read 2”) is aligned to a reverse strand. In some embodiments, the system may be configured to determine a first FSC corresponding to the fragmentation site 302A as the subsequence 304A “CACCTC” of the reference genome. The system may be configured to determine a second FSC corresponding to the fragmentation site 302B as the reverse complement of the subsequence 304B because the second read is aligned to the reverse strand. Thus, the system determines the second FSC to be the reverse complement of “GCGCCT” which is “AGGCGC”.

FIG. 4 shows an example PPM 400 that may encode information about an FSC distribution of a cfDNA sample, according to some embodiments of the technology described herein. The PPM 400 may be generated by any suitable computing device. In some embodiments, the PPM 400 may be the data structure 146 generated by the FSC recognition module 104 of the cancer detection system 100 described herein with reference to FIG. 1A-FIG. 1B.

As shown in FIG. 4, the PPM 400 includes a row for each of the possible dinucleotides that may occur in an FSC context. There are 16 possible dinucleotides which are as follows: “AA”, “AC”, “AG”, “AT”, “CA”, “CC”, “CG”, “CT”, “GA”, “GC”, “GG”, “GT”, “TA”, “TC”, “TG”, and “TT”. The PPM 400 includes 5 columns are each associated with a pair of positions in the FSC context where a dinucleotide would occur. The positions are labeled in FIG. 4 with respect to a fragmentation site. The first column from the left labeled (−3, −2) represents the furthest pair of positions preceding the fragmentation site (i.e., external to the cfDNA fragment), the second column labeled (−2, −1) represents the closest pair positions preceding the fragmentation site, the third column labeled (−1, +1) represents the pair of positions separated by the fragmentation site, the fourth column labeled (+1, +2) represents the closest pair of positions following the fragmentation site (i.e., internal to the cfDNA fragment), and the fifth column labeled (+2, +3) represents the further pair of positions following the fragmentation site. Each element in the PPM 400 indicates a probability of a particular dinucleotide occurring at a particular pair of positions in the FSC. For example, the top left element indicates a probability that the dinucleotide “AA” occurs at the furthest pair of positions external to the fragmentation site in an FSC is 0.046. The top right element indicates a probability that the dinucleotide “AA” occurs at the furthest pair of positions internal to the fragmentation site in an FSC is 0.106.

FIG. 5A illustrates classifications 502A, 502B that may be determined by the cancer detection system 100 (described herein with reference to FIG. 1A-FIG. 1B) using one or more trained machine learning models 500, according to some embodiments of the technology described herein. The classifications 502A, 502B may be determined by any suitable computing device. In some embodiments, the classifications 502A, 502B may be determined by the cancer detection module 106 of the cancer detection system 100. As shown in FIG. 5A, the cancer detection system 100 determines, using the data structure 146 encoding information about an FSC distribution of a cfDNA sample from a subject, a cancer classification 502A indicating whether the subject has cancer and, if so, a classification 502B of a tissue of origin of the cancer.

In some embodiments, the system may be configured to determine both of the classifications 502A, 502B using a single machine learning model. The machine learning model may be a multi-class model that is trained to output results indicating both of the classifications 502A, 502B. The machine learning model may be trained to output a classification result for each of multiple classes including: a first class indicating whether the cfDNA sample is cancerous and multiple tissue of origin classes. The cancer detection system 100 may be configured to use the data structure 146 or a derivative thereof as a set of features to provide as input to the machine learning model to obtain output. The output may indicate: (1) a first cancer classification result indicating whether the cfDNA sample is cancerous; and (2) a set of tissue of origin classification results that each indicate whether cancer, if present in the cfDNA sample, originates from a particular tissue. For example, each classification result may be a binary value. As another example, each classification result may be an indication of likelihood (e.g., a probability value) that the input set of features belongs to a class. The system may determine the cancer classification 502A using the indication of likelihood that the cfDNA sample is cancerous. If the system determines that the cfDNA sample is likely cancerous (e.g., by determining that an output probability value for the cancer classification is greater than 0.5), then the system may determine the cancer classification 502A to be that the cfDNA sample represented by the data structure 146 is cancerous. Otherwise, the system may determine the cancer classification 502A to be that the cfDNA sample is not cancerous. If the cfDNA sample is cancerous, the system may determine the tissue of origin classification 502B based on indications of likelihoods determined for the tissue of origin classifications. The system may identify the tissue of origin class with the greatest likelihood (e.g., the highest probability value) to be the tissue of origin classification 502B.

In some embodiments, the machine learning model(s) 500 may include multiple machine learning models to use in determining the cancer classification 502A and the tissue of origin classification 502B. The multiple machine learning models may include a first machine learning model trained to output the cancer classification 502A and a second machine learning model trained to output the tissue of origin classification 502B. The system may be configured to use the data structure 146 or a derivative thereof as a set of features to provide as input to both machine learning models. The first machine learning model may output an indication of the cancer classification 502A (e.g., a binary value or a probability that the cfDNA sample is cancerous). The second machine learning model may output an indication of which tissue cancer in the cfDNA sample originates from (e.g., as binary values associated with tissue of origin classes or probability values associated with tissue of origin classes).

Examples of machine learning model(s) 500 that may be used by the system include a support vector machine (SVM) model, a random forest model, an isolation forest model, a gradient boosting classification model, an extremely randomized trees model, a logistic regression model, and/or a neural network model. A machine learning model may be trained by applying a learning algorithm to a training dataset. In some embodiments, the machine learning model may be trained by applying a supervised learning algorithm to a labeled training dataset. The labeled training dataset may include multiple labeled sets of features (e.g., data structures encoding information about FSC distributions or derivatives thereof). Each set of features may be labeled as cancerous or healthy. Each set of features may further be labeled with a tissue of origin. The machine learning model may be trained by: (1) providing the sets of features as input to the machine learning model to obtain corresponding outputs; (2) determining a difference between the outputs and the labels of the sets of features; and (3) updating parameters of the machine learning model based on a difference between the outputs and the labels. As an illustrative example, the machine learning model may be trained by applying a stochastic gradient descent algorithm to the labeled training data set. In some embodiments, the machine learning model may be trained by applying an unsupervised learning algorithm to an unlabeled training dataset. For example, a clustering algorithm may be applied to a set of data structures or derivatives thereof to obtain clusters (e.g., a cluster representing cancerous cfDNA samples and a cluster representing non-cancerous cfDNA samples, and/or clusters associated with respective tissues of origin). The clusters may be used to perform classification (e.g., distance-based classification or anomaly detection).

FIG. 5B illustrates determination of distance measurements using the data structure 146 encoding information about an FSC distribution, according to some embodiments of the technology described herein. The distance measurements may be used to determine classifications for a cfDNA sample associated with the data structure 146. For example, the distance measurements may be used to determine similarity measurements between the data structure 146 and reference sets of reference data structures (e.g., associated with non-cancerous and cancerous cfDNA samples, and/or associated with different tissues of origin). As another example, the distance measurements may be used to determine feature values to provide as input to a machine learning model. The determination of distance measurements illustrated by FIG. 5B may be performed by any suitable computing device. In some embodiments, the determination may be performed by the cancer detection module 106 of the cancer detection system 100 described herein with reference to FIG. 1A-FIG. 1B.

As illustrated in FIG. 5B, in some embodiments, the system may be configured to project the data structure 146 into a projection space 510 to obtain the projection 516. The system may be configured to project the data structure 146 into the projection space 510 using any suitable technique. In some embodiments, the system may be configured to project the data structure 146 into a vector of real number values. The system may be configured to normalize the real number values such that they sum to 1. In some embodiments, the system may be configured to project the data structure to a vector of 2 values, 3 values, 4 values, 5 values, 6 values, 7 values, 8 values, 9 values, 10 values, or another suitable number of values. For example, the projection space 510 may be a 4-dimensional space and the system may project the data structure 146 into the projection space 510 as a vector of 4 real number values. In some embodiments, projecting the data structure 146 into the projection space 510 may involve applying a transformation to values of the data structure 146 and/or a numerical vector obtained from the data structure 146. For example, projecting the data structure 146 may involve applying a logarithmic function to values in the data structure 146 and/or vector values to obtain the projection 516. As another example, projecting the data structure 146 into the projection space 510 may involve using principal component analysis (PCA) vectors to project the data structure 146 into the projection space 510 thereby obtaining the projection 516.

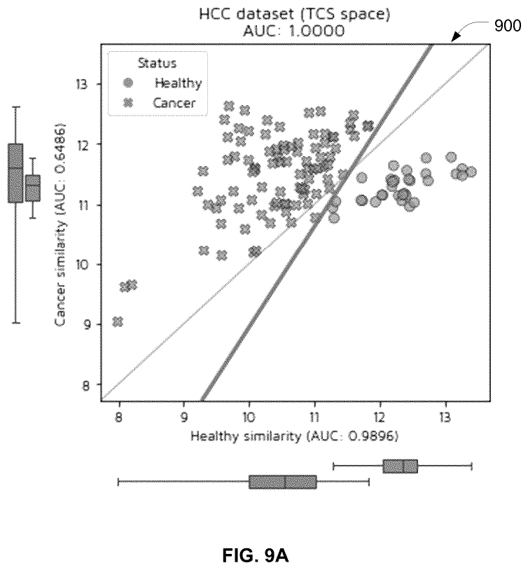

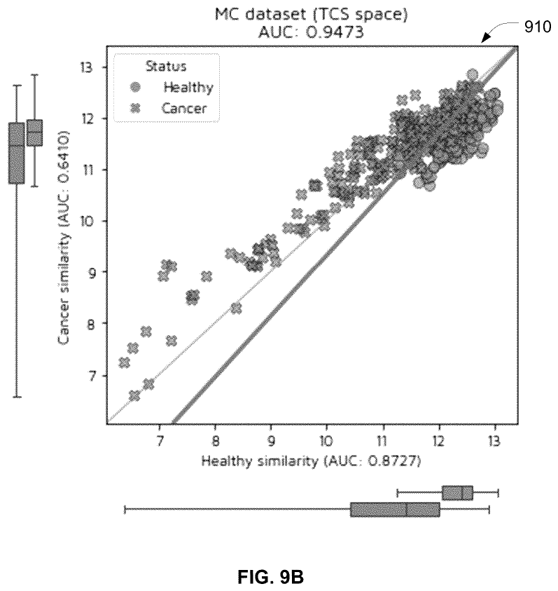

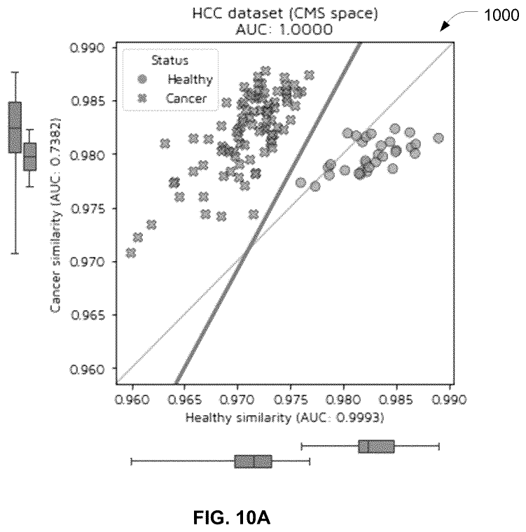

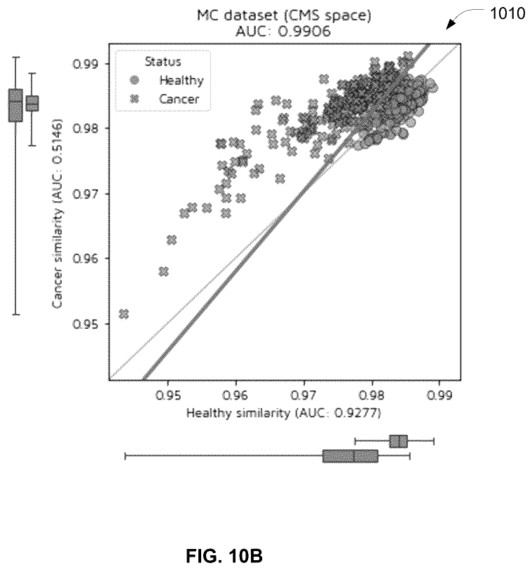

In some embodiments, the system may be configured to project the data structure 146 by applying a transformation to the data structure 146. For example, the system may apply a total class similarity (TCS) similarity transformation (described herein with reference to FIG. 9A-FIG. 9B) to the data structure 146. As another example, the system may apply a class Mahalanobis similarity (CMS) transformation (described herein with reference to FIG. 10A-FIG. 10B). As another example, the system may combine TCS and CMS transformations to obtain a joint transformation of the data structure 146.

As illustrated in FIG. 5B, there are two reference sets of points in the projection space 510 for which distance measurements are determined. The two reference sets of points are a set of cancerous points 520 and a set of non-cancerous points 522. The set of cancerous points 520 may be obtained by projecting data structures encoding information about FSC distributions of cfDNA samples that are known to be cancerous. The set of non-cancerous points 522 may be obtained by projecting data structures encoding information about FSC distributions of cfDNA samples that are known to be non-cancerous. The system may be configured to project the data structure 146 into the projection space 510 using the same projection technique used to project data structures into the projection space to obtain the set of cancerous points 520 and the set of non-cancerous points 522. In some embodiments, a reference set of points may be obtained by: (1) determining projections of a set of data structures into the projection space 510; and (2) filtering the projections to obtain the reference set of points. For example, the projections may be filtered by removing outlier points. For example, an outlier point may be a point that is greater than a threshold distance away from a centroid of the projections.

As illustrated in FIG. 5B, in some embodiments, the system may be configured to determine: (1) a distance measurement 518 between the projection 516 of the data structure 146 and the set of cancerous points 520; and (2) a distance measurement 519 between the projection 516 of the data structure 146 and the set of non-cancerous points 522. The system may be configured to determine the distance measurements 518, 519 using any suitable measure of distance. Examples of measures of distance that may be used by the system include Jensen-Shannon distance (JSD), Mahalanobis distance, Euclidean distance, Manhattan distance, Cosine distance, Chebyshev distance, Bray-Curtis distance, Canberra distance, Correlation distance, Minkowski distance (e.g., with p=1.5). In one example implementation, the system may be configured to determine the distance measurements 518, 519 by determining the JSD between the projection 516 and each of the sets of points 520, 522. In another example implementation, the system may be configured to determine the distance measurements 518, 519 by determining the Mahalanobis distance between the projection 516 and the sets' points 520, 522.

The system may be configured to determine a distance measurement between the projection 516 and a set of points (e.g., cancerous points 520 or non-cancerous points 522) using a measure of distance. As illustrated in the example of FIG. 5B, in some embodiments, the system may be configured to determine a distance measurement between the projection 516 and a set of points by determining a distance measurement between the projection 516 and a centroid of the set of points in the projection space 510. In some embodiments, the system may be configured to determine a distance measurement between the projection 516 and a set of reference points by: (1) determining a set of distance measurements between the projection 516 and each point in the set of reference points (e.g., each of the cancerous points 520 or each of the non-cancerous points 522); and (2) determining the distance measurement between the projection 516 using the set of distance measurements. For example, the system may determine the distance measurement to be a mean of the set of distance measurements, the maximum of the set of distance measurements (i.e., the furthest point in the reference set of points), or the minimum of the set of distance measurements (i.e., the closest point in the reference set of points).

Although the example of FIG. 5B involves projection of the data structure 146 into a projection space 510, in some embodiments, the system may be configured to determine distance measurements without projecting the data structure 146. For example, the system may determine distance measurement(s) between the data structure 146 and reference set(s) of data structures. The system may thus determine the distance measurements without projecting the data structure 146 into any projection space.

In some embodiments, the system may be configured to use a distance measurement (e.g., distance measurement 518 or distance measurement 519) to determine a similarity measurement between the data structure 146 and data structures represented by a reference set of points (e.g., cancerous points 520 or non-cancerous points 522). The measure of similarity may be derived from a measure of distance used to determine the distance measurement. As an illustrative example, the measure of similarity S (x, y) may be determined from a measure of distance D(x, y) as S(x, y)=D(x, y)^{-E} where the value of the exponent E is customized for the specific measure of distance. For example, the exponent E may be equal to 0.01 when Mahalanobis distance is used as the measure of distance D(x, y) used to determine the distance measurement. As another example, the exponent E may be equal to 5 when JSD is used as the measure of distance D(x, y) to determine the distance measurement. In some embodiments, the measure of similarity may compress the measure of distance into a smaller range while preserving the relative order of points to facilitate classification and visualization. Similarity measurements derived from the distance measurements may be used to perform classification. For example, the system may: (1) determine a first similarity measurement using the distance measurement 518 and a second similarity measurement using the distance measurement 519; and (2) classify the cfDNA sample associated with the data structure 146 as cancerous or non-cancerous using the first and second similarity measurements (e.g., by classifying the cfDNA sample into a class corresponding to the higher similarity measurement or using the first and second similarity measurements to determine feature values to input to a machine learning model).

In some embodiments, the system may further be configured to determine distance measurements between the projection 516 of the data structure 146 and sets of reference points associated with different tissues of origin (e.g., to use the distance measurements for determining a classification of tissue of origin of cancerous cfDNA fragments in the cfDNA sample associated with the data structure 146). The system may be configured to use distance measurement techniques described herein with reference to FIG. 5B to determine the distance measurements. In some embodiments, the system may be configured to determine similarity measurements using the distance measurements (e.g., to use for determining a classification of tissue of origin of cancerous cfDNA fragments in the cfDNA sample).

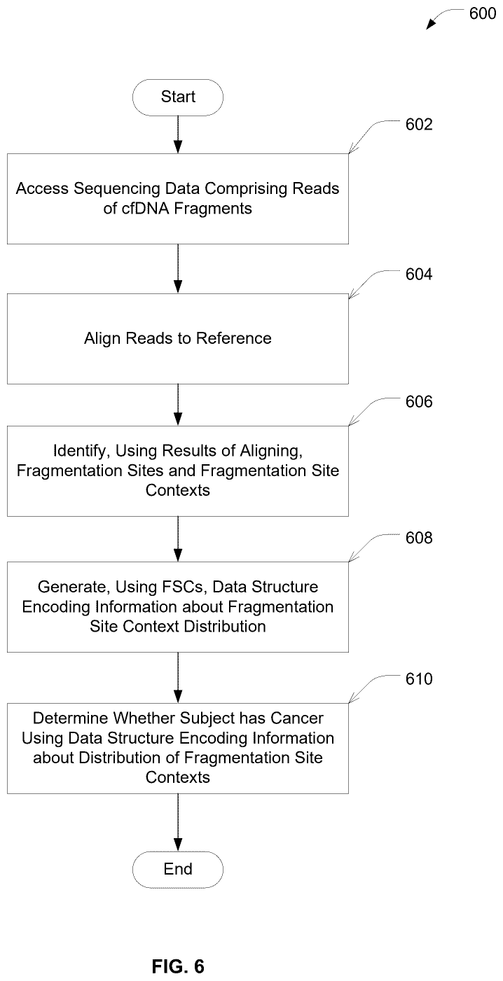

FIG. 6 illustrates an example process 600 for determining whether a subject has cancer by analyzing fragmentation in a cfDNA sample obtained from the subject, according to some embodiments of the technology described herein. Process 600 may be performed by any suitable computing device. In some embodiments, process 600 may be performed by cancer detection system 100 described herein with reference to FIG. 1A-FIG. 1B.

Process 600 begins at block 602, where the system accesses sequencing data comprising reads of cfDNA fragments. The sequencing data may have been previously obtained by performing sequencing on the cfDNA sample (e.g., by sequencing platform 110 described herein with reference to FIG. 1A). Each of the reads may be a sequence of nucleotides of a cfDNA fragment. In some embodiments, the reads may include reads aligned to a reverse strand. Thus, for a particular cfDNA fragment, the reads may include a first read aligned to a reverse strand and a second read aligned to a forward strand.

The system may be configured to access the sequencing data using any suitable technique. In some embodiments, the system may be configured to access the sequencing data from data storage of the system. In some embodiments, the system may be configured to access the sequencing data from external data storage (e.g., storage integrated with a sequencing platform, or an external database). For example, the system may access the sequencing data by querying an external database.

At block 604, the system aligns the reads to a reference. In some embodiments, the system may be configured to align the reads to a human reference genome (e.g., for determining whether a human subject has cancer). The system may be configured to align the reads to the reference by using an alignment algorithm. Example techniques of aligning the reads to the reference are described herein with reference to the alignment module 102 of cancer detection system 100.

At block 606, the system identifies, using results of the aligning performed at block 604, fragmentation sites and corresponding fragmentation site contexts. In some embodiments, the system may be configured to identify fragmentation sites using results of the aligning by identifying, as the fragmentation sites, points in the reference that are between a portion of the reference aligned with a read and a portion of the reference that is not aligned with the read. In other words, the system may be configured to identify fragmentation sites as points that lie between a portion of the reference internal to a cfDNA fragment and a portion of the reference external to the cfDNA fragment. In some embodiments, the system may be configured to indicate the fragmentation sites with coordinate values. For example, the system may indicate a fragmentation site as a coordinate in the reference (e.g., a nucleotide position in the reference). Examples illustrating identification of fragmentation sites are described herein with reference to FIG. 2A and FIG. 3.

The system may be configured to identify FSCs corresponding to the fragmentation sites using various techniques. In some embodiments, the system may be configured to identify FSCs corresponding to a fragmentation site by identifying a nucleotide subsequence of the reference spanning the fragmentation site (e.g., a hexamer of the reference centered at the fragmentation site), and using the nucleotide subsequence to determine the FSC corresponding to the fragmentation site. For example, the system may determine the nucleotide subsequence to be the FSC. As another example, the system may determine a reverse complement of the nucleotide subsequence as the FSC (e.g., where the read was aligned to a reverse strand). An example of identifying an FSC using a nucleotide subsequence of the reference is described herein with reference to FIG. 2A and FIG. 3. In some embodiments, the system may be configured to identify FSCs corresponding to a fragmentation site by identifying a nucleotide subsequence of a read marking the fragmentation site, and using the identified nucleotide subsequence to determine the FSC corresponding to the fragmentation site. In such embodiments, the FSC may consist of a nucleotide subsequence that is internal to a cfDNA fragment. An example of identifying an FSC using a nucleotide subsequence of a read is described herein with reference to FIG. 2B.

At block 608, the system generates, using the identified FSCs, a data structure (e.g., a PPM) encoding information about the FSC distribution of the cfDNA sample. An example data structure is described herein with reference to FIG. 4. In some embodiments, the data structure may indicate probabilities of different nucleotide sequences occurring at various FSC positions. For example, the data structure may be a matrix in which each element indicates a probability of a particular nucleotide sequence (e.g., a dinucleotide) occurring at a particular set of one or more FSC positions. The system may be configured to generate the data structure by determining the probabilities and storing the probabilities in the data structure. The system may be configured to determine the probability of a particular nucleotide sequence (e.g., a mononucleotide or dinucleotide) occurring at a particular set of FSC position(s) by: (1) determining the number of occurrences of the particular nucleotide sequence at the particular FSC position across all the identified FSCs; and (2) dividing the number by the total number of FSCs to obtain the probability. As an illustrative example, the system may generate a matrix in which each row represents a dinucleotide and each column represents a pair of FSC positions. Each element in the matrix may indicate the probability of a dinucleotide associated with the element's row occurring at a pair of FSC positions represented by the element's column.

At block 610, the system determines whether the subject has cancer using the data structure (e.g., the PPM) encoding information about the FSC distribution of the cfDNA sample. In some embodiments, the system may be configured to use a machine learning model to determine a classification of whether the cfDNA sample is cancerous (e.g., as described herein with reference to FIG. 5A). Example machine learning models that may be used by the system are described herein. The system may be configured to use the data structure to generate an input (e.g., an input set of features) and provide the input to the machine learning model to obtain output indicating the classification. In some embodiments, the system may be configured to determine whether the subject has cancer by determining similarity measurements between the data structure and one or more sets of reference data structures (e.g., a reference set of data structures representing known cancerous cfDNA samples and/or a reference set of data structures representing known non-cancerous cfDNA samples). The system may be configured to determine the similarity measurements by determining distance measurements between the data structure and the set(s) of reference data structures (e.g., as described herein with reference to FIG. 5B).

In some embodiments, the system may be configured to determine a classification of whether the subject has cancer by projecting the data structure into a projection space (e.g., as described herein with reference to FIG. 5B). The system may be configured to perform classification (e.g., by using a machine learning model and/or by determining similarity measurements) using the projection of the data structure. In some embodiments, the system may be configured to use the data structure as features to provide as input to a machine learning model.

In some embodiments, the system may be configured to determine whether the subject has cancer by determining whether the subject has a particular one of multiple cancer types using the data structure encoding information about the distribution of fragmentation site contexts of the cfDNA sample. The system may be configured to determine whether the subject has a particular one of multiple types of cancers using the data structure encoding information about the distribution of fragmentation site contexts of the cfDNA sample by: (1) determining a measure of similarity (e.g., a measure of distance) between the data structure and multiple cancer-specific sets of data structures to obtain multiple similarity measurements for the multiple cancer types, and (2) determining whether the subject has a particular one of the multiple cancer types using the multiple similarity measurements for the multiple cancer-specific sets of data structures. The multiple cancer-specific sets of data structures each encoding information about fragmentation site context distributions of cancerous cfDNA samples of a respective one of the multiple cancer types. For example, the system may determine a measure of distance between a PPM and sets of PPMs corresponding to the multiple cancer types. Accordingly, the system may be configured to classify the cfDNA sample into one of the multiple cancer types as part of determining whether the subject has cancer.

In some embodiments, the system may be configured to use a machine learning model to determine a classification of the cfDNA sample into one of multiple cancer types. Example machine learning models that may be used by the system are described herein. The system may be configured to use the data structure to generate an input (e.g., an input set of features) and provide the input to the machine learning model to obtain output indicating the classification. In some embodiments, the system may be configured to determine a classification of the cfDNA sample into one of multiple cancer types by determining similarity measurements between the data structure and sets of reference data structures (e.g., each representing known cancerous cfDNA samples of a particular one of the multiple cancer types). The system may be configured to determine the similarity measurements by determining distance measurements between the data structure and the sets of reference data structures (e.g., as described herein with reference to FIG. 5B). In some embodiments, the system may be configured to determine the classification of the cfDNA sample in one of multiple cancer types by projecting the data structure into a projection space (e.g., as described herein with reference to FIG. 5B). The system may be configured to perform classification (e.g., by using a machine learning model and/or by determining similarity measurements) using the projection of the data structure. For example, the system may be configured to use the data structure as features to provide as input to a machine learning model. As another example, the system may be configured to determine similarity measurements between the projection of the data structure and sets of projections of reference data structures associated with the multiple cancer types (e.g., by determining similarity measurements between the projection of the data structure and centroids of the sets of projections of reference data structures).

In some embodiments, the system may be further configured to determine a tissue of origin of the cancer when the subject is determined to have cancer. The system may be configured to determine the tissue of origin by determining, using the data structure, a classification of the cfDNA sample into one of multiple classes representing respective tissues of origin. In some embodiments, the system may be configured to determine the classification using a machine learning model (e.g., the same machine learning model used to determine whether the subject has cancer or a different machine learning model). The system may be configured to generate input using the data structure and provide the input to the machine learning model to obtain output indicating a tissue of origin classification. In some embodiments, the system may be configured to determine the classification by determining similarity measurements between the data structure and sets of reference data structures associated with different tissues of origin (e.g., generated FSCs of cfDNA samples with cancer from the different tissues of origin). The system may be configured to determine a tissue of origin classification for cancer in the cfDNA sample using the similarity measurements (e.g., by selecting a tissue of origin class associated with the greatest similarity measurement).

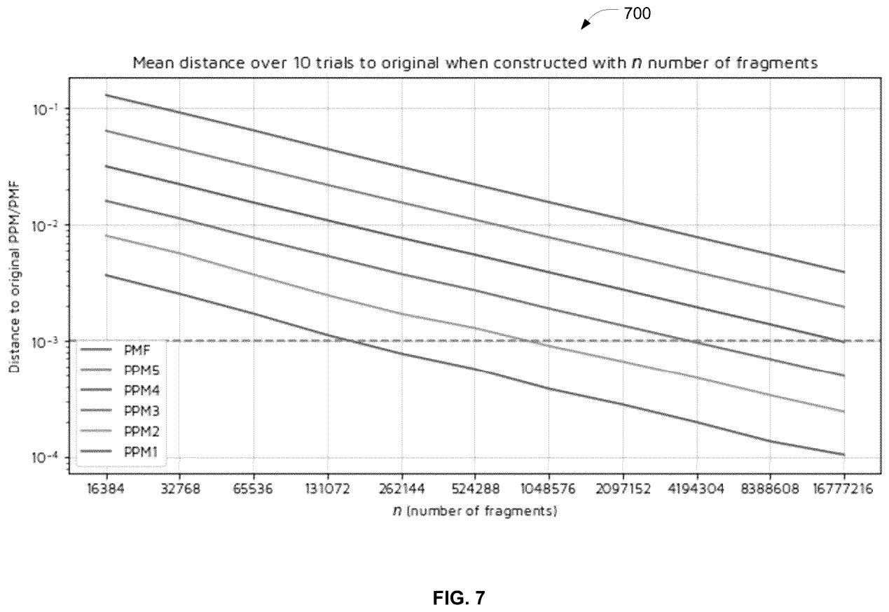

FIG. 7 shows a graph 700 of mean distance between original PMF/PPMs and PMF/PPMs generated using FSC contexts identified using reads of cfDNA fragments. In the example of FIG. 7, the FSC contexts are hexamers centered at fragmentation sites. In FIG. 7, PPM1 refers to a PPM built with mononucleotide frequencies. PPM2 refers to a PPM built with dinucleotide frequencies. PPM3 refers to a PPM built with trinucleotide frequencies. PPM4 refers to a PPM built with tetranucleotide frequencies. PPM5 refers to a PPM built with pentanucleotide frequencies. As shown in the graph 700, the larger PPMs and the PMF require more fragments to obtain an estimate that is close (distance<0.001) to the original PPMs and the original PMF. The PMF requires more reads of fragments than any PPM size. Accordingly, the PMF requires a greater depth of sequencing to obtain more reads that can be used to generate the PMF.

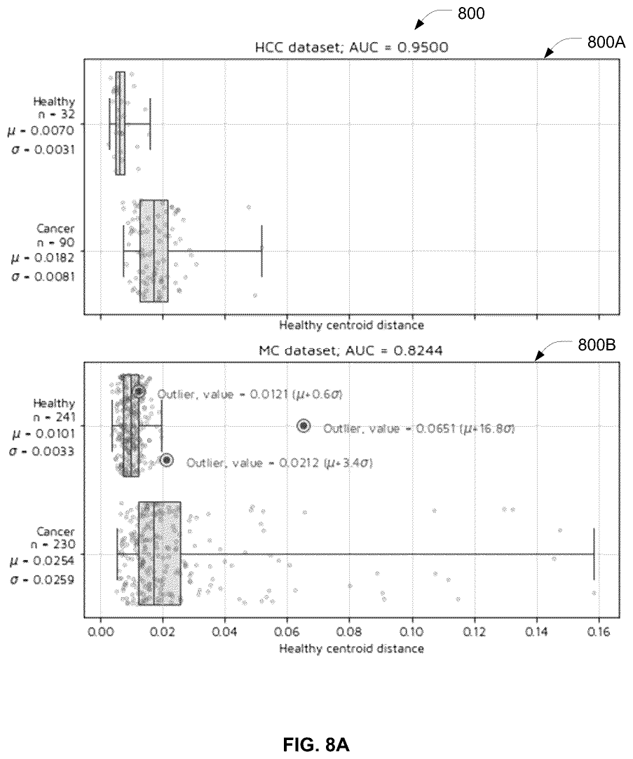

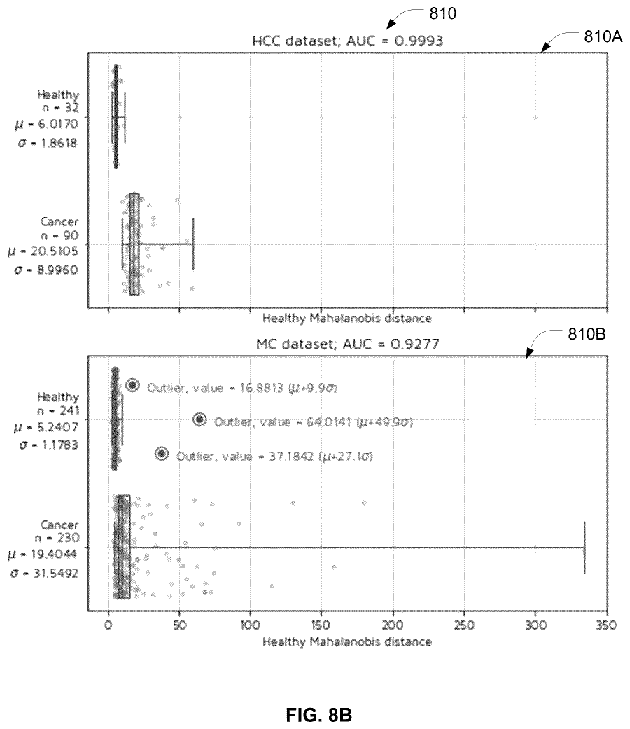

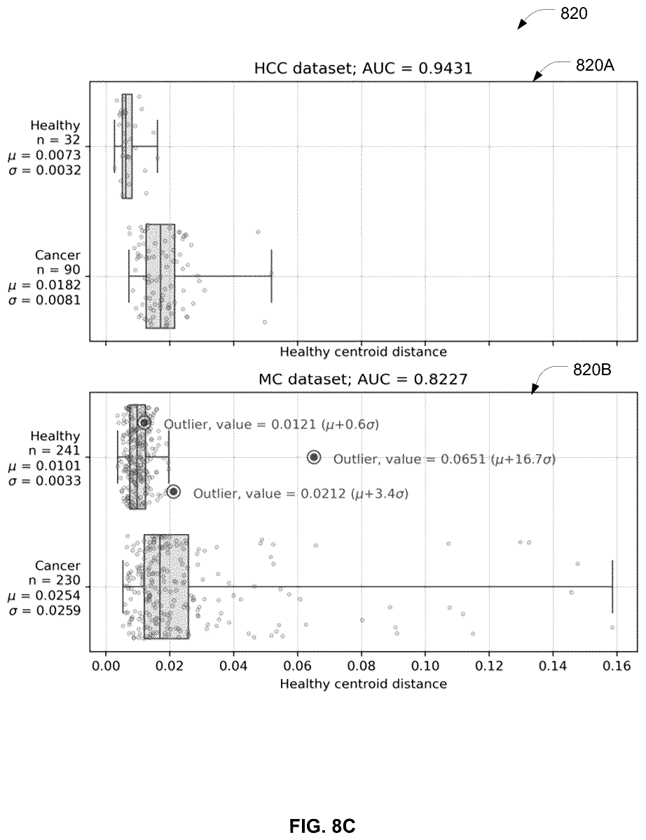

FIG. 8A shows graphs 800 illustrating distances of non-cancerous PPMs from a centroid of the non-cancerous PPMs and distances of cancerous PPMs from a centroid of the non-cancerous PPMs for two different datasets. The graph 800A shows centroid distances determined using points in the HCC dataset from Jiang et al., 2015—doi: 10.1073/pnas. 1500076112 and the graph 800B shows centroid distances determined using points in the MC dataset from Cristiano et al., 2019—doi: 10.1038/s41586-019-1272-6. The HCC dataset has sequencing data obtained from sequencing 32 non-cancerous (“healthy”) cfDNA samples and 90 early stage liver cancer cfDNA samples. The MC dataset has sequencing data obtained from sequencing 241 healthy cfDNA samples and 230 cancerous cfDNA samples. The MC dataset has higher healthy variability than the HCC dataset, which results in a larger distance between healthy samples. The measure of distance in the example of FIG. 8A is JSD. A receiver operating characteristic (ROC) curve illustrates classification performance of a machine learning model for different classification thresholds. The area under the ROC curve (AUC) provides an aggregate measure of performance across all the classification thresholds. As shown in FIG. 8A, the healthy centroid distance can be a biomarker as it provides a high AUC of 0.95 for the HCC dataset and an AUC of 0.8244 for the MC dataset.

FIG. 8B shows graphs 810 illustrating distances of noncancerous PPMs to a distribution of the noncancerous PPMs and distances of cancerous PPMs to a distribution of the non-cancerous PPMs. The measure of distance in the example of FIG. 8B is Mahalanobis distance (MD). The graph 810A shows MDs determined using points in the HCC dataset and the graph 810B shows MDs determined using points in the MC dataset. The measure of distance in the example of FIG. 8B is Mahalanobis distance. The MD based classification has an AUC of 0.9993 for samples in the HCC dataset and an AUC of 0.9277 for samples in the MC dataset.

FIG. 8C shows graphs 820 illustrating distances of non-cancerous PPMs from a centroid of the non-cancerous PPMs and distances of cancerous PPMs from a centroid of the non-cancerous PPMs for two different datasets. The graph 820A shows centroid distances determined using points in the HCC dataset and the graph 820B shows centroid distances determined using points in the MC dataset. The measure of distance in the example of FIG. 8C is JSD. In this example, the healthy centroid used for determining a distance from a given point is computed using all other points that belong to the healthy class in the dataset. A receiver operating characteristic (ROC) curve illustrates classification performance of a machine learning model for different classification thresholds. The area under the ROC curve (AUC) provides an aggregate measure of performance across all the classification thresholds. As shown in FIG. 8C, the healthy centroid distance can be a biomarker as it provides a high AUC of 0.9431 for the HCC dataset and an AUC of 0.8227 for the MC dataset.

FIG. 9A shows a graph 900 illustrating total class similarity (TCS) transformation of PPMs generated from samples in the HCC dataset. FIG. 9B shows a graph 910 of TCS transformation of PPMs generated from samples in the MC dataset. The transformation step uses a similarity measure based on JSD: S(u, v)=JSD(u, v)⁻⁵. For each PPM, a similarity is calculated between the PPM and all other PPMs. The similarities are summed for the two classes (i.e., cancerous and noncancerous). A value of 1 is added to each total and then a base 10 logarithm is taken of the resulting value. In some embodiments, classification can be performed in the TCS space using a machine learning model. As shown in FIG. 9A and FIG. 9B, the AUC for classification of samples from the HCC dataset using TCS is 1 and the AUC for classification of samples from the MC dataset using TCS is 0.9473.

FIG. 10A shows a graph 1000 of class Mahalanobis similarity (CMS) transformation of PPMs generated from samples in the HCC dataset. FIG. 10B shows a graph 1010 of CMS transformation of PPMs generated from samples in the MC dataset. The CMS is performed by finding both classes' centroids and covariance matrices, and adding 10⁻⁷ to each diagonal element of the covariance matrices to allow for inversion. Then the Mahalanobis distance (MD) is calculated between each point and the various classes, and converted to a similarity by the equation S=D^{-0.01}. When comparing a point to its own class, the centroid and covariance matrix are computed after removing the point so that the distance is from the point to the distribution defined by the other points in the class. In some embodiments, classification can be performed in the space using a machine learning model. In some embodiments, classification can be formed in the space using a distance-based classification. As shown in FIG. 10A and FIG. 10B, the AUC for classification of samples from the HCC dataset using CMS is 1 and the AUC for classification performed of samples from the MC dataset using CMS is 0.9906.

FIG. 11 shows a graph 1100 illustrating use of a combination of TCS and CMS transformations (referred to as “TCMS”) to perform classification on samples in the HCC dataset, and a graph 1110 illustrating use of TCMS to perform classification on samples in the MC dataset. When combining the two transformations' results, the TCS values are scaled to be in the same range as the CMS values to facilitate classification. As illustrated by the graphs 1100 and 1110, TCMS produces better classification in a cross-validation experiment relative to use of TCS transformation (as described with reference to FIG. 9A and FIG. 9B) and CMS transformation (as described with reference to FIG. 10A and FIG. 10B). The cross-validation AUC for classification of samples from the HCC dataset using TCMS is 1 while the cross-validation AUC for classification of samples from the MC dataset using TCMS is 0.9905.

The classification performance illustrated in FIG. 11 is better than other classification techniques. An entropy-based classification technique described in Jiang et al., 2020—doi: 10.1158/2159-8290.CD-19-0622 has an AUC of 0.86 on a dataset of 38 healthy individuals and 34 with liver cancer. A classification technique GALYFRE described in Budhraja et al., 2023 - doi: 10.1126/scitranslmed.abm6863 that uses a random forest model has an AUC of 0.91 on a combination of two datasets including data from the MC dataset. Another classification technique referred to as DELFI described in Cristiano et al., 2019—doi: 10.1038/s41586-019-1272-6 has an AUC of 0.94 for classifying samples from the MC dataset. Accordingly, the TCMS based classification illustrated in FIG. 11 performs better than these existing techniques (e.g., as indicated by AUC).

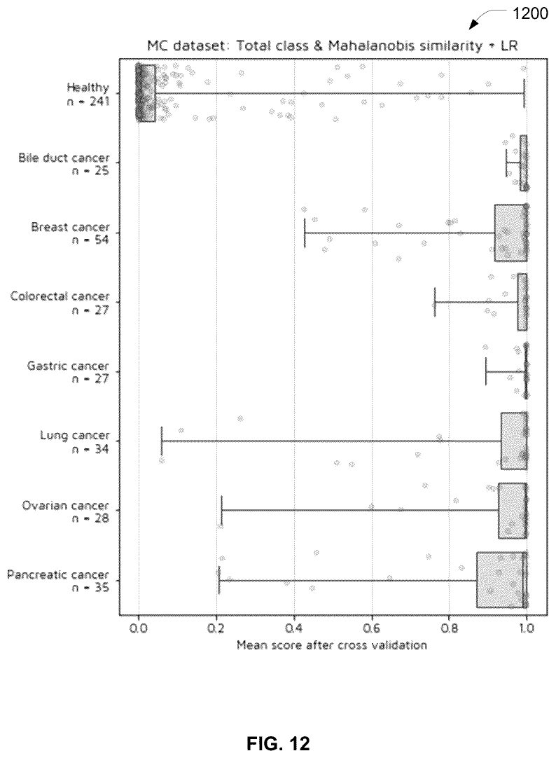

FIG. 12 shows a graph 1200 illustrating performance of cancer classification using TCMS. The scores in FIG. 12 reflect cancer detection scores across difference types of cancer. This shows that the detection performance is consistent across the different types of cancer. The system classified the cfDNA samples using data structures (e.g., PPMs) generated for the samples by: (1) applying the TCMS transformation to the data structures; and (2) performing classification using the transformations (e.g., using a machine learning model or a distance-based classification). The classification for all tissue of origin classes had a Q1 mean score that is greater than 0.8.

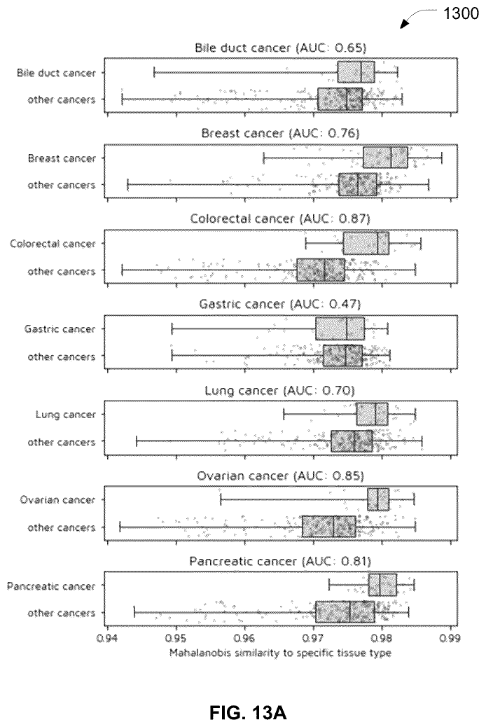

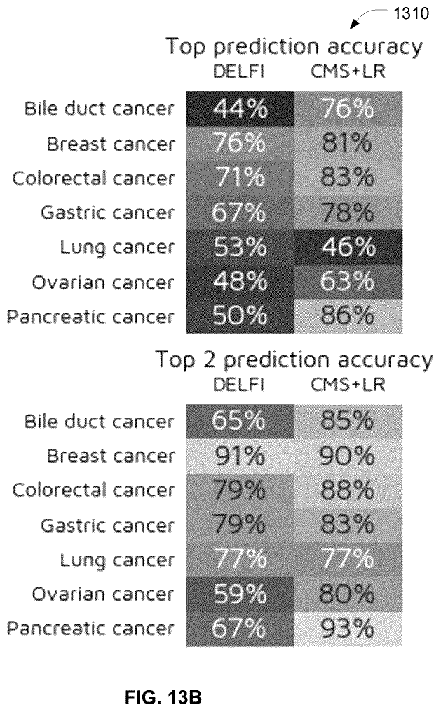

FIG. 13A shows a graph 1300 illustrating performance of tissue of origin classification on the MC dataset using Mahalanobis similarity. In the example of FIG. 13, the system classified cancerous cfDNA samples into one of the following tissues of origin: bile duct cancer, breast cancer, colorectal cancer, gastric cancer, lung cancer, ovarian cancer, and pancreatic cancer. As shown in the graph 1300, the cancers of a given tissue generally tend to be more similar to each other than other cancers. Accordingly, the Mahalanobis similarity may be used to determine a tissue of origin for a cancerous cfDNA sample (e.g., by using the Mahalanobis similarity or a similarity measure derived therefrom).

FIG. 13B shows a graph 1310 comparing the tissue of origin prediction accuracy of an example embodiment to the DELFI classification technique described in Cristiano et al., 2019—doi: 10.1038/s41586-019-1272-6. As shown in FIG. 13B, the example embodiment's performance (in the column labeled “CMS+LR”) is comparable or better than the DELFI classification technique. The example embodiment has significantly better prediction accuracy for bile duct, ovarian, and pancreatic cancers than the DELFI classification technique.

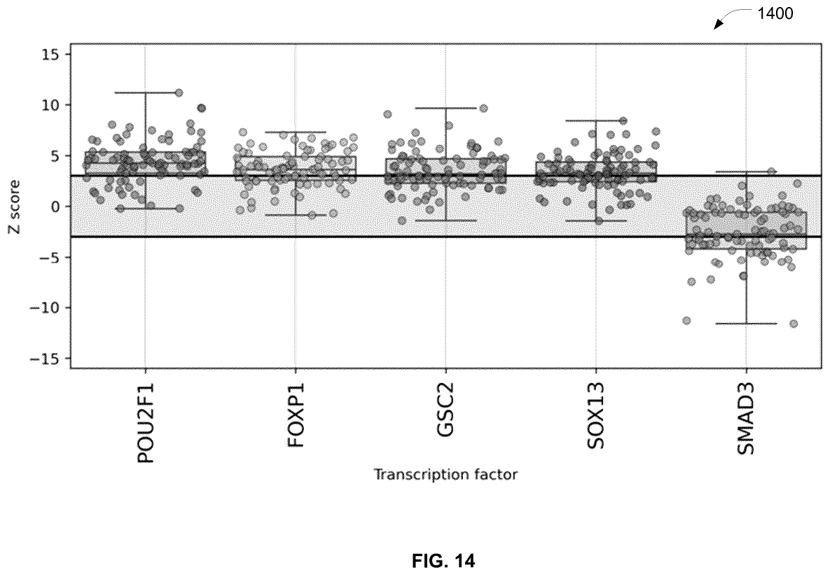

FIG. 14 shows a graph 1400 illustrating performance of cancer detection using information about fragmentation site contexts corresponding to transcription factor binding sites (TFBSs), according to some embodiments of the technology described herein. The analyzed TFBS motifs were obtained from the JASPAR dataset in Nucleic Acids Research, Volume 52, Issue D1, 5 Jan. 2024, Pages D174-D182. Hexamer PMF data within the HCC dataset was compared against each motif. For each motif, the optimal scoring 6-base pair window was identified for the healthy data within the HCC dataset. A position weight matrix (PWM) was generated by comparing the optimal hexamer motif (represented as a PPM) against a PPM generated from frequencies of mononucleotides or dinucleotides (as appropriate) found in the genome. These per-TFBS PWMs were then used to score each hexamer PMF: a TFBS score is the sum of each hexamer's frequency in the PMF multiplied by the PWM score for that hexamer. 755 PWMs were evaluated for the TFBS motifs obtained from the JASPAR dataset. For each sample in the HCC dataset, a TFBS PWM score was calculated for each TFBS. Recursive feature elimination was performed for each TFBS to rank the TFBS PWM scores in terms of how informative they were for detecting cancer. The graph 1400 shows results for the top 5 TFBSs. All 5 of the TFBSs are associated with liver cancer (as described below). The scores illustrated in the graph 1400 are the TFBS PWM scores converted to Z scores for cancer samples of the top 5 TFBSs. Healthy samples in the HCC dataset were used to obtain the mean and standard deviation for standardization. These results illustrate that the fragmentation site contexts effectively distinguish between cancerous and healthy samples.

The top 5 TFBSs are: POU2F1, FOXP1, GSC2, SOX13, SMAD3. Each of these TFBSs have been associated with liver cancer. POU2F1 promotes growth and metastasis of hepatocellular carcinoma through the FAT1 signaling pathway as described in Am J Cancer Res. 2017 Aug. 1; 7(8):1665-1679. PMID: 28861323; PMCID: PMC5574939. POU2F1 over-expression correlates with poor prognoses and promotes cell growth and epithelial-to-mesenchymal transition in hepatocellular carcinoma as described in Oncotarget. 2017 Jul. 4; 8(27):44082-44095. doi: 10.18632/oncotarget.17296. PMID: 28489585; PMCID: PMC5546464. Downregulation of FOXP1 inhibits cell proliferation in hepatocellular carcinoma by inducing G1/S phase cell cycle arrest as described in Int J Mol Sci. 2016 Sep. 8; 17(9):1501. doi: 10.3390/ijms17091501. PMID: 27618020; PMCID: PMC5037778. GSC2 has high homology to Goosecoid (GSC) as described in PMID 9700206 (Gottlieb et al., 1998). GSC promotes the metastasis of hepatocellular carcinoma by modulating the epithelial-mesenchymal transition as described in PLOS One. 2014 Oct. 24; 9(10):e109695. doi: 10.1371/journal.pone.0109695. PMID: 25343336; PMCID: PMC4208742. SOX13 regulates cancer stem-like properties and tumorigenicity in hepatocellular carcinoma cells as described in Am J Cancer Res. 2021 Mar. 1; 11(3):760-772. PMID: 33791152; PMCID: PMC7994154. SOX13 promotes hepatocellular carcinoma metastasis by transcriptionally activating Twist1 as described in Lab Invest. 2020 November; 100(11):1400-1410. doi: 10.1038/s41374-020-0445-0. Epub 2020 May 27. PMID: 32461589. SMAD3 reduces susceptibility to hepatocarcinoma by sensitizing hepatocytes to apoptosis through downregulation of Bcl-2 as described in Cancer Cell. 2006 June; 9(6):445-57. doi: 10.1016/j.ccr.2006.04.025. PMID: 16766264; PMCID: PMC2708973.

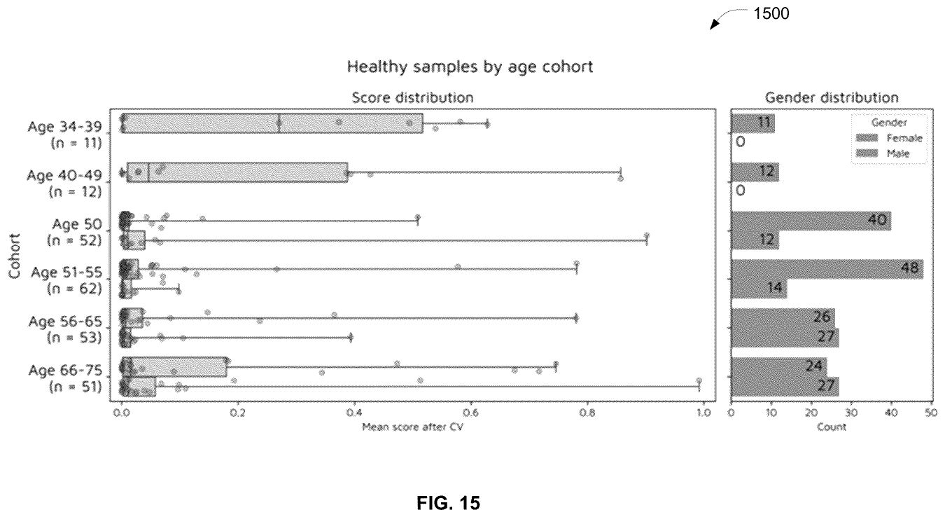

FIG. 15 shows a graph 1500 illustrating scores generated for healthy samples by processing fragmentation site contexts for the healthy samples using a trained machine learning model, according to some embodiments of the technology described herein. The scores plotted in the graph 1500 were computed by performing cross validation on the MC dataset. The MC dataset has age and gender metadata that was used to generate the data illustrated in the graph 1500. As can be seen in the graph 1500, the cohort of samples that are less than the age of 50 are all female. The scores obtained for the under 50 cohort have elevated scores relative to the other age groups. The elevated scores may be from pregnant donors who were referred for a Pap smear, with fetal cfDNA detected as an abnormality. This indicates that the fragmentation site contexts provide biologically relevant signals, as opposed to signals resulting purely from effects of data processing (e.g., batch processing effects).

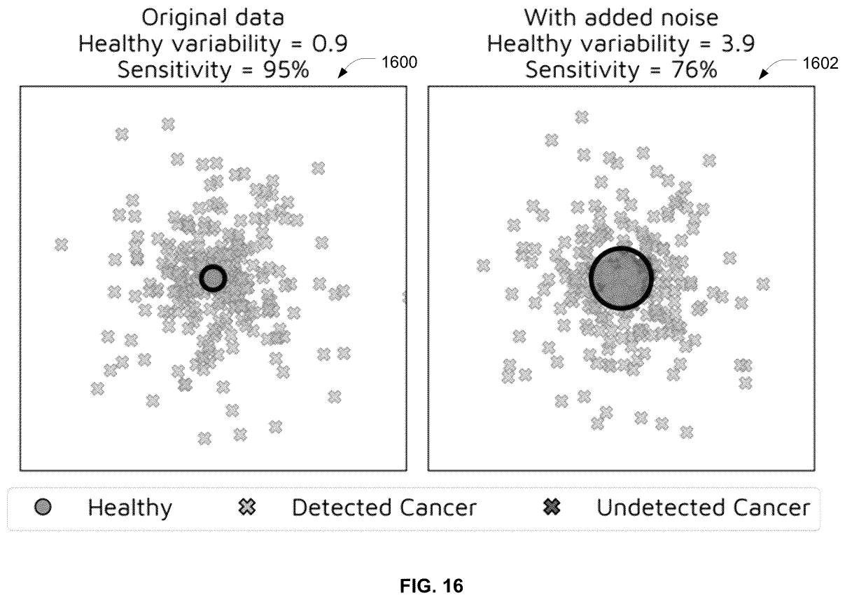

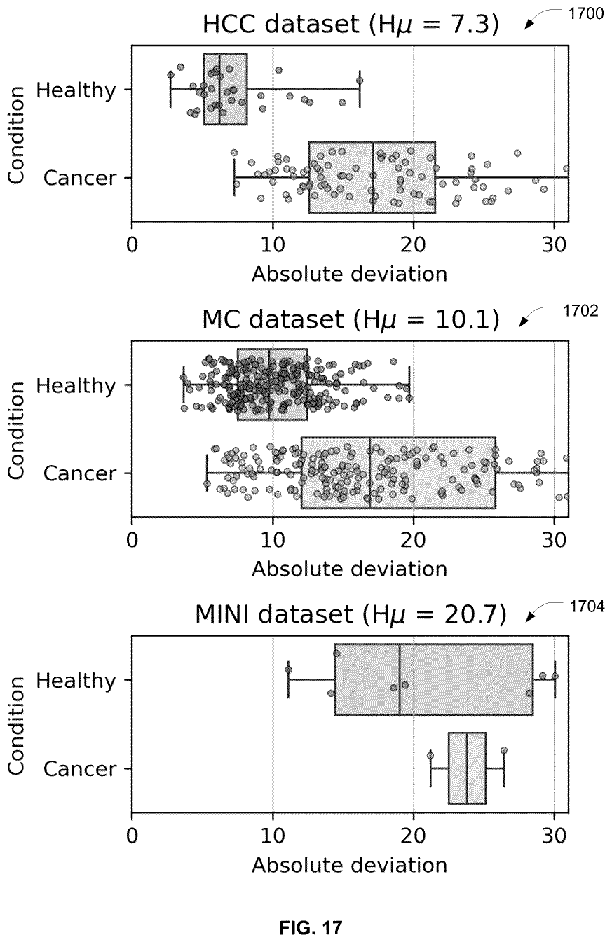

FIG. 16 illustrates graphs 1600, 1602 showing the impact of added noise on sensitivity determined for synthetic 2D data generated using a random number generator, according to some embodiments of the technology described herein. In this synthetic data, the distance of a point from the center of a graph is analogous to the healthy centroid distance (HCD) for a PPM as shown in FIGS. 8A, 8C, and 17. The graph 1600 illustrates points in the originally generated data and the graph 1602 illustrates the same points with added noise. A large black circle in graphs 1600 and 1602 illustrates the radius at which 100% specificity would be determined. The graphs 1600, 1602 illustrate that HCD is an effective metric in differentiating healthy and cancer samples. In the original data, there is a variability of 0.9 and a sensitivity of 95%. In the data with added noise, there is a variability of 3.9 and a sensitivity of 76%. This shows that variability has a significant impact on accuracy. Thus, it is important to minimize variability (e.g., due to sampling techniques, environmental factors, and/or other causes).

FIG. 17 illustrates graphs 1700, 1702, 1704 of healthy centroid distances determined for PPMs generated from samples in various datasets, according to some embodiments of the technology described herein. The graphs 1700, 1702, 1704 show absolute deviation for samples, which is the healthy centroid distance multiplied by 1000. Hu indicates the mean of the healthy samples' absolute deviation within the respective dataset. Graph 1700 shows absolute deviation for samples from the HCC dataset. Graph 1702 shows absolute deviation for samples from the MC dataset. Graph 1704 shows absolute deviation for samples from a dataset called “MINI”. The MINI dataset contains 8 healthy samples and 2 breast cancer samples. The healthy exclusion criteria used to obtain the samples in the MINI dataset are as follows:
1. No older than 30 years old
2. BMI between 20 and 29
3. No people who are or think they might be pregnant
4. No history of smoking
5. No more than 6 drinks per week of alcohol
6. No history of cancer
7. No current (acute or chronic) disease
8. No known infectious/non-infectious illness w/in past 3 months that required a medical appointment
9. No transplant recipients

The breast cancer samples in the MINI dataset are as follows:
1. ductal carcinoma, age 74, female, Stage III-A
2. lobular carcinoma, age 85, female, Stage II-A

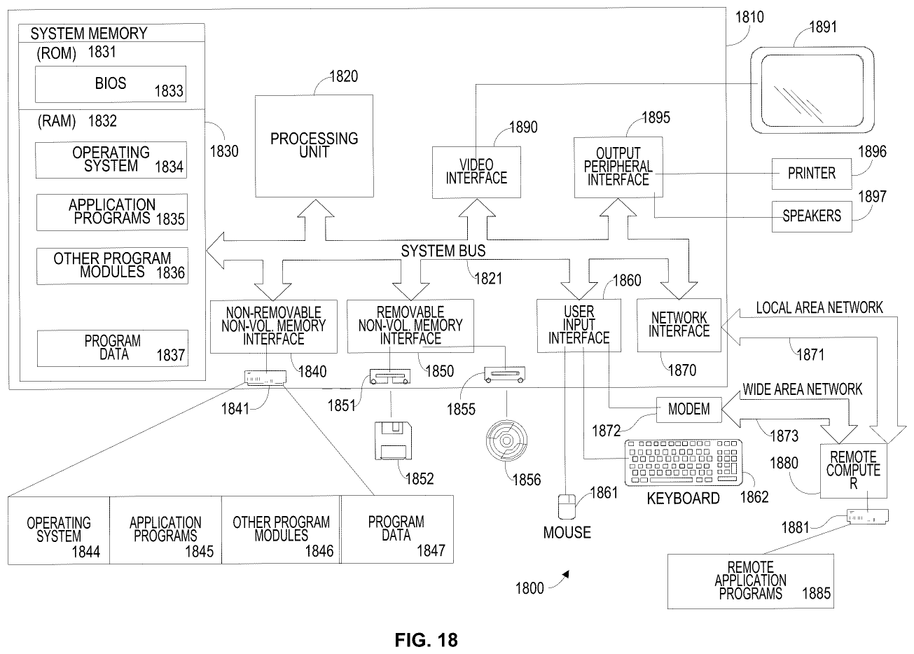

FIG. 18 shows a block diagram of an exemplary computing device, in accordance with some embodiments of the technology described herein. The computing system environment 1800 is only one example of a suitable computing environment and is not intended to suggest any limitation as to the scope of use or functionality of the technology described herein.

The technology described herein is operational with numerous other general purpose or special purpose computing system environments or configurations. Examples of well-known computing systems, environments, and/or configurations that may be suitable for use with the technology described herein include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The technology described herein may also be practiced in distributed computing environments where tasks are performed by remote processing devices that are linked through a communications network. In a distributed computing environment, program modules may be located in both local and remote computer storage media including memory storage devices.

With reference to FIG. 18, an exemplary system for implementing the technology described herein includes a general purpose computing device in the form of a computer 1810. Components of computer 1810 may include, but are not limited to, a processing unit 1820, a system memory 1830, and a system bus 1821 that couples various system components including the system memory to the processing unit 1820. The system bus 1821 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 1810 typically includes a variety of computer readable media. Computer readable media can be any available media that can be accessed by computer 1810 and includes both volatile and nonvolatile media, removable and non-removable media. By way of example, and not limitation, computer readable media may comprise computer storage media and communication media. Computer storage media includes volatile and nonvolatile, removable, and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 1810. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 1830 includes computer storage media in the form of volatile and/or nonvolatile memory such as read only memory (ROM) 1831 and random access memory (RAM) 1832. A basic input/output system 1833 (BIOS), containing the basic routines that help to transfer information between elements within computer 1810, such as during start-up, is typically stored in ROM 1831. RAM 1832 typically contains data and/or program modules that are immediately accessible to and/or presently being operated on by processing unit 1820. By way of example, and not limitation, FIG. 18 illustrates operating system 1834, application programs 1835, other program modules 1836, and program data 1837.

The computer 1810 may also include other removable/non-removable, volatile or nonvolatile computer storage media. By way of example only, FIG. 18 illustrates a hard disk drive 1841 that reads from or writes to non-removable, nonvolatile magnetic media, a flash drive 1851 that reads from or writes to a removable, nonvolatile memory 1852 such as flash memory, and an optical disk drive 1855 that reads from or writes to a removable, nonvolatile optical disk 1856 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 1841 is typically connected to the system bus 1821 through a non-removable memory interface such as interface 1840, and magnetic disk drive 1851 and optical disk drive 1855 are typically connected to the system bus 1821 by a removable memory interface, such as interface 1850.

The drives and their associated computer storage media described above and illustrated in FIG. 18, provide storage of computer readable instructions, data structures, program modules and other data for the computer 1810. In FIG. 18, for example, hard disk drive 1841 is illustrated as storing operating system 1844, application programs 1845, other program modules 1846, and program data 1847. Note that these components can either be the same as or different from operating system 1834, application programs 1835, other program modules 1836, and program data 1837. Operating system 1844, application programs 1845, other program modules 1846, and program data 1847 are given different numbers here to illustrate that, at a minimum, they are different copies. An actor may enter commands and information into the computer 1810 through input devices such as a keyboard 1862 and pointing device 1861, commonly referred to as a mouse, trackball, or touch pad. Other input devices (not shown) may include a microphone, joystick, game pad, satellite dish, scanner, or the like. These and other input devices are often connected to the processing unit 1820 through a user input interface 1860 that is coupled to the system bus, but may be connected by other interface and bus structures, such as a parallel port, game port or a universal serial bus (USB). A monitor 1891 or other type of display device is also connected to the system bus 1821 via an interface, such as a video interface 1890. In addition to the monitor, computers may also include other peripheral output devices such as speakers 1897 and printer 1896, which may be connected through an output peripheral interface 1895.

The computer 1810 may operate in a networked environment using logical connections to one or more remote computers, such as a remote computer 1880. The remote computer 1880 may be a personal computer, a server, a router, a network PC, a peer device, or other common network node, and typically includes many or all of the elements described above relative to the computer 1810, although only a memory storage device 1881 has been illustrated in FIG. 18. The logical connections depicted in FIG. 18 include a local area network (LAN) 1871 and a wide area network (WAN) 1873, but may also include other networks. Such networking environments are commonplace in offices, enterprise-wide computer networks, intranets, and the Internet.

When used in a LAN networking environment, the computer 1810 is connected to the LAN 1871 through a network interface or adapter 1870. When used in a WAN networking environment, the computer 1810 typically includes a modem 1872 or other means for establishing communications over the WAN 1873, such as the Internet. The modem 1872, which may be internal or external, may be connected to the system bus 1821 via the actor input interface 1860, or other appropriate mechanism. In a networked environment, program modules depicted relative to the computer 1810, or portions thereof, may be stored in the remote memory storage device. By way of example, and not limitation, FIG. 18 illustrates remote application programs 1885 as residing on memory device 1881. It will be appreciated that the network connections shown are exemplary and other means of establishing a communications link between the computers may be used.

Having thus described several aspects of at least one embodiment of the technology described herein, it is to be appreciated that various alterations, modifications, and improvements will readily occur to those skilled in the art. Such alterations, modifications, and improvements are intended to be part of this disclosure, and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Further, it should be appreciated that a computer may be embodied in any of a number of forms, such as a rack-mounted computer, a desktop computer, a laptop computer, a tablet computer, a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. A computer-readable storage medium includes any computer memory configured to store software, for example, the memory of any computing device such as a smart phone, a laptop, a desktop, a rack-mounted computer, or a server (e.g., a server storing software distributed by downloading over a network, such as an app store)). As used herein, the term “computer-readable storage medium” encompasses only a non-transitory computer-readable medium that can be considered to be a manufacture (i.e., article of manufacture) or a machine. Alternatively, or additionally, aspects of the technology described herein may be embodied as a computer readable medium other than a computer-readable storage medium, such as a propagating signal.

The terms “program” or “software” are used herein in a generic sense to refer to any type of computer code or set of processor-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor, but may be distributed in a modular fashion among a number of different computers or processors to implement various aspects of the technology described herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

### EXAMPLE EMBODIMENTS

In some embodiments, the techniques described herein relate to a method for determining whether a subject has cancer by analyzing fragmentation in a cell-free deoxyribonucleic acid (cfDNA) sample obtained from the subject, the method including: using at least one computer hardware processor to perform: accessing sequencing data, the sequencing data previously obtained from sequencing the cfDNA sample, the sequencing data including a plurality of reads of cfDNA fragments; aligning the plurality of reads to a reference; identifying, using results of aligning the plurality of reads to the reference, fragmentation sites of the cfDNA sample and nucleotide subsequences of the reference corresponding to the fragmentation sites; determining fragmentation site contexts of the cfDNA sample using the nucleotide subsequences of the reference corresponding to the fragmentation sites; generating, using the plurality of fragmentation site contexts, a data structure encoding information about a distribution of fragmentation site contexts of the cfDNA sample; and determining whether the subject has cancer using the data structure encoding information about the distribution of fragmentation site contexts of the cfDNA sample.

In some embodiments, the techniques described herein relate to a method, wherein identifying, using results of aligning the plurality of reads to the reference, the nucleotide subsequences of the reference corresponding to the fragmentation sites includes: identifying, for each of the fragmentation sites, a nucleotide subsequence in the reference that spans the fragmentation site.

In some embodiments, the techniques described herein relate to a method, wherein identifying, for each of the fragmentation sites, a nucleotide subsequence in the reference that spans the fragmentation site includes: identifying a hexamer spanning the fragmentation site as the nucleotide subsequence.

In some embodiments, the techniques described herein relate to a method, wherein identifying, for each of the fragmentation sites, a nucleotide subsequence in the reference that spans the fragmentation site includes: identifying the nucleotide subsequence as one or more nucleotides preceding the fragmentation site and one or more nucleotides following the fragmentation site.

In some embodiments, the techniques described herein relate to a method, wherein generating the data structure encoding information about the distribution of fragmentation site contexts of the cfDNA sample includes generating a data structure indicating, for each of a plurality of nucleotide sequences of a fixed length, estimated probabilities of the nucleotide sequence occurring at a plurality of fragmentation site context positions.

In some embodiments, the techniques described herein relate to a method, wherein generating, using the plurality of fragmentation site contexts, the data structure encoding information about the distribution of fragmentation site contexts of the cfDNA sample includes: generating a position probability matrix (PPM) that indicates, for each of the plurality of nucleotide sequences of the fixed length, estimated probabilities of the nucleotide sequence occurring at the plurality of fragmentation site context positions.

In some embodiments, the techniques described herein relate to a method, wherein the plurality of nucleotides of the fixed length are dinucleotides. In some embodiments, the techniques described herein relate to a method, further including determining a tumor's tissue of origin using the data structure encoding information about the distribution of fragmentation site contexts of the cfDNA sample.

In some embodiments, the techniques described herein relate to a method, wherein determining the fragmentation site contexts of the cfDNA sample using the nucleotide subsequences of the reference corresponding to the fragmentation sites includes: determining a first one of the nucleotide subsequences corresponding to a first fragmentation site to be a first fragmentation site context; and determining a reverse complement of a second one of the nucleotide subsequences corresponding to a second fragmentation site as a second fragmentation site context of the fragmentation site contexts.

In some embodiments, the techniques described herein relate to a method, wherein determining whether the subject has cancer using the data structure encoding information about the distribution of fragmentation site contexts of the cfDNA sample includes: determining a measure of similarity between the data structure and a first plurality of data structures encoding information about fragmentation site context distributions of cancerous cfDNA samples to obtain a first similarity measurement; and determining whether the subject has cancer using the first similarity measurement.

In some embodiments, the techniques described herein relate to a method, wherein determining whether the subject has cancer using the data structure encoding information about the distribution of fragmentation site contexts of the cfDNA sample includes: determining the measure of similarity between the data structure and a second plurality of data structures encoding information about fragmentation site context distributions of non-cancerous cfDNA samples to obtain a second similarity measurement; and determining whether the subject has cancer using the first similarity measurement and/or the second similarity measurement.

In some embodiments, the techniques described herein relate to a method, wherein determining the measure of similarity between the data structure and the first plurality of data structures encoding information about fragmentation site context distributions of cancerous cfDNA samples includes: determining a first distance measurement between the data structure and the first plurality of data structures encoding information about fragmentation site context distributions of cancerous cfDNA; and determining the first similarity measurement using the first distance measurement.

In some embodiments, the techniques described herein relate to a method, further including determining the measure of similarity between the data structure and a second plurality of data structures encoding information about fragmentation site context distributions of non-cancerous cfDNA samples by: determining a second distance measurement between the data structure and the second plurality of data structures encoding information about fragmentation site context distributions of non-cancerous cfDNA samples; and determining the second similarity measurement using the second distance measurement.

In some embodiments, the techniques described herein relate to a method,, wherein determining the first and second distance measurements includes: determining a measure of distance between the data structure and the first plurality of data structures to obtain the first distance measurement; and determining the measure of distance between the data structure and the second plurality of data structures to obtain the second distance measurement. In some embodiments, the techniques described herein relate to a method, wherein the measure of distance is Jensen-Shannon distance (JSD). In some embodiments, the techniques described herein relate to a method, wherein the measure of distance is Mahalanobis distance.

In some embodiments, the techniques described herein relate to a method,, wherein determining whether the subject has cancer using the data structure encoding information about the distribution of fragmentation site contexts of the cfDNA sample includes: projecting the data structure into a projection space to obtain a projection of the data structure; and determining whether the subject has cancer using the projection of the data structure.

In some embodiments, the techniques described herein relate to a method, wherein determining whether the subject has cancer using the projection of the data structure includes: determining whether the subject has cancer using projections into the projection space of: a first plurality of data structures encoding information about fragmentation site context distributions of cancerous cfDNA samples; and/or a second plurality of data structures encoding information about fragmentation site context distributions of non-cancerous cfDNA samples.

In some embodiments, the techniques described herein relate to a method, further including: when it is determined that the subject has cancer, determining the cancer's tissue of origin using the data structure encoding information about the fragmentation site context distribution of the cfDNA sample.

In some embodiments, the techniques described herein relate to a method, wherein determining the cancer's tissue of origin using the data structure encoding information about the fragmentation site context distribution of the cfDNA sample includes: determining similarity measurements between the data structure encoding information about the distribution of fragmentation site contexts of the cfDNA sample and a plurality of reference data structure sets each associated with a tissue of origin and including data structures encoding information about distributions of fragmentation site contexts of cfDNA samples with cancer from the tissue of origin.

In some embodiments, the techniques described herein relate to a method, further including: determining an intervention for the subject based on the cancer's tissue of origin.

In some embodiments, the techniques described herein relate to a method, further including: when it is determined that the patient has cancer, triggering administration of treatment to the patient.

In some embodiments, the techniques described herein relate to a method, wherein determining whether the subject has cancer using the data structure encoding information about the distribution of fragmentation site contexts of the cfDNA sample includes: determining whether the subject has a particular one of multiple cancer types using the data structure encoding information about the distribution of fragmentation site contexts of the cfDNA sample.

In some embodiments, the techniques described herein relate to a method, wherein determining whether the subject has a particular one of multiple types of cancers using the data structure encoding information about the distribution of fragmentation site contexts of the cfDNA sample includes: determining a measure of similarity between the data structure and multiple cancer-specific sets of data structures to obtain multiple similarity measurements for the multiple cancer types, the multiple cancer-specific sets of data structures each encoding information about fragmentation site context distributions of cancerous cfDNA samples of a respective one of the multiple cancer types; and determining whether the subject has a particular one of the multiple cancer types using the multiple similarity measurements for the multiple cancer-specific sets of data structures.

In some embodiments, the techniques described herein relate to a system for determining whether a subject has cancer by analyzing fragmentation in a cell-free deoxyribonucleic acid (cfDNA) sample obtained from the subject, the system including: at least one computer hardware processor; and at least one non-transitory computer-readable storage medium storing instructions that, when executed by the at least one computer hardware processor, cause the at least one computer hardware processor to perform: accessing sequencing data, the sequencing data previously obtained from sequencing the cfDNA sample, the sequencing data including a plurality of reads of cfDNA fragments; aligning the plurality of reads to a reference; identifying, using results of aligning the plurality of reads to the reference, fragmentation sites of the cfDNA sample and nucleotide subsequences of the reference corresponding to the fragmentation sites; determining fragmentation site contexts of the cfDNA sample using the nucleotide subsequences of the reference corresponding to the fragmentation sites; generating, using the plurality of fragmentation site contexts, a data structure encoding information about a distribution of fragmentation site contexts of the cfDNA sample; and determining whether the subject has cancer using the data structure encoding information about the distribution of fragmentation site contexts of the cfDNA sample.

In some embodiments, the techniques described herein relate to a non-transitory computer-readable storage medium storing instructions that, when executed by at least one computer hardware processor, cause the at least one computer hardware processor to perform a method for determining whether a subject has cancer by analyzing fragmentation in a cell-free deoxyribonucleic acid (cfDNA) sample obtained from the subject, the method including: accessing sequencing data, the sequencing data previously obtained from sequencing the cfDNA sample, the sequencing data including a plurality of reads of cfDNA fragments; aligning the plurality of reads to a reference; identifying, using results of aligning the plurality of reads to the reference, fragmentation sites of the cfDNA sample and nucleotide subsequences of the reference corresponding to the fragmentation sites; determining fragmentation site contexts of the cfDNA sample using the nucleotide subsequences of the reference corresponding to the fragmentation sites; generating, using the plurality of fragmentation site contexts, a data structure encoding information about a distribution of fragmentation site contexts of the cfDNA sample; and determining whether the subject has cancer using the data structure encoding information about the distribution of fragmentation site contexts of the cfDNA sample.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A-only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every clement specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method for determining whether a subject has cancer by analyzing fragmentation in a cell-free deoxyribonucleic acid (cfDNA) sample obtained from the subject, the method comprising:
using at least one computer hardware processor to perform:
accessing sequencing data, the sequencing data previously obtained from sequencing the cfDNA sample, the sequencing data comprising a plurality of reads of cfDNA fragments;
aligning the plurality of reads to a reference;
identifying, using results of aligning the plurality of reads to the reference, fragmentation sites of the cfDNA sample and nucleotide subsequences of the reference corresponding to the fragmentation sites;
determining fragmentation site contexts of the cfDNA sample using the nucleotide subsequences of the reference corresponding to the fragmentation sites;
generating, using the plurality of fragmentation site contexts, a data structure encoding information about a distribution of fragmentation site contexts of the cfDNA sample; and
determining whether the subject has cancer using the data structure encoding information about the distribution of fragmentation site contexts of the cfDNA sample.

2. The method of claim 1, wherein identifying, using results of aligning the plurality of reads to the reference, the nucleotide subsequences of the reference corresponding to the fragmentation sites comprises:
identifying, for each of the fragmentation sites, a nucleotide subsequence in the reference that spans the fragmentation site.

3. The method of claim 2, wherein identifying, for each of the fragmentation sites, a nucleotide subsequence in the reference that spans the fragmentation site comprises:
identifying a hexamer spanning the fragmentation site as the nucleotide subsequence.

4. The method of claim 1, wherein generating the data structure encoding information about the distribution of fragmentation site contexts of the cfDNA sample comprises generating a data structure indicating, for each of a plurality of nucleotide sequences of a fixed length, estimated probabilities of the nucleotide sequence occurring at a plurality of fragmentation site context positions.

5. The method of claim 4, wherein generating, using the plurality of fragmentation site contexts, the data structure encoding information about the distribution of fragmentation site contexts of the cfDNA sample comprises:
generating a position probability matrix (PPM) that indicates, for each of the plurality of nucleotide sequences of the fixed length, estimated probabilities of the nucleotide sequence occurring at the plurality of fragmentation site context positions.

6. The method of claim 4, wherein the plurality of nucleotides of the fixed length are dinucleotides.

7. The method of claim 1, further comprising determining a tumor's tissue of origin using the data structure encoding information about the distribution of fragmentation site contexts of the cfDNA sample.

8. The method of claim 1, wherein determining the fragmentation site contexts of the cfDNA sample using the nucleotide subsequences of the reference corresponding to the fragmentation sites comprises:
determining a first one of the nucleotide subsequences corresponding to a first fragmentation site to be a first fragmentation site context; and
determining a reverse complement of a second one of the nucleotide subsequences corresponding to a second fragmentation site as a second fragmentation site context of the fragmentation site contexts.

9. The method of claim 1, wherein determining whether the subject has cancer using the data structure encoding information about the distribution of fragmentation site contexts of the cfDNA sample comprises:
determining a measure of similarity between the data structure and a first plurality of data structures encoding information about fragmentation site context distributions of cancerous cfDNA samples to obtain a first similarity measurement; and
determining whether the subject has cancer using the first similarity measurement.

10. The method of claim 9, wherein determining whether the subject has cancer using the data structure encoding information about the distribution of fragmentation site contexts of the cfDNA sample comprises:
determining the measure of similarity between the data structure and a second plurality of data structures encoding information about fragmentation site context distributions of non-cancerous cfDNA samples to obtain a second similarity measurement; and
determining whether the subject has cancer using the first similarity measurement and/or the second similarity measurement.

11. The method of claim 10, wherein:
determining the measure of similarity between the data structure and the first plurality of data structures comprises:
determining a measure of distance between the data structure and the first plurality of data structures to obtain a first distance measurement; and
determining the first similarity measurement using the first distance measurement; and
determining the measure of similarity between the data structure and the second plurality of data structures comprises:
determining the measure of distance between the data structure and the second plurality of data structures to obtain a second distance measurement; and
determining the second similarity measurement using the second distance measurement.

12. The method of claim 11, wherein the measure of distance is Mahalanobis distance.

13. The method of claim 1, wherein determining whether the subject has cancer using the data structure encoding information about the distribution of fragmentation site contexts of the cfDNA sample comprises:
projecting the data structure into a projection space to obtain a projection of the data structure; and
determining whether the subject has cancer using the projection of the data structure and projections of:
a first plurality of data structures encoding information about fragmentation site context distributions of cancerous cfDNA samples; and/or
a second plurality of data structures encoding information about fragmentation site context distributions of non-cancerous cfDNA samples.

14. The method of claim 1, further comprising:
when it is determined that the subject has cancer, determining the cancer's tissue of origin using the data structure encoding information about the fragmentation site context distribution of the cfDNA sample.

15. The method of claim 14, wherein determining the cancer's tissue of origin using the data structure encoding information about the fragmentation site context distribution of the cfDNA sample comprises:
determining similarity measurements between the data structure encoding information about the distribution of fragmentation site contexts of the cfDNA sample and a plurality of reference data structure sets each associated with a tissue of origin and comprising data structures encoding information about distributions of fragmentation site contexts of cfDNA samples with cancer from the tissue of origin.

16. The method of claim 15, further comprising:
determining an intervention for the subject based on the cancer's tissue of origin.

17. The method of claim 1, further comprising:
when it is determined that the patient has cancer, triggering administration of treatment to the patient.

18. The method of claim 1, wherein determining whether the subject has cancer using the data structure encoding information about the distribution of fragmentation site contexts of the cfDNA sample comprises:
determining whether the subject has a particular one of multiple cancer types using the data structure encoding information about the distribution of fragmentation site contexts of the cfDNA sample.

19. A system for determining whether a subject has cancer by analyzing fragmentation in a cell-free deoxyribonucleic acid (cfDNA) sample obtained from the subject, the system comprising:
at least one computer hardware processor; and
at least one non-transitory computer-readable storage medium storing instructions that, when executed by the at least one computer hardware processor, cause the at least one computer hardware processor to perform:
accessing sequencing data, the sequencing data previously obtained from sequencing the cfDNA sample, the sequencing data comprising a plurality of reads of cfDNA fragments;
aligning the plurality of reads to a reference;
identifying, using results of aligning the plurality of reads to the reference, fragmentation sites of the cfDNA sample and nucleotide subsequences of the reference corresponding to the fragmentation sites;
determining fragmentation site contexts of the cfDNA sample using the nucleotide subsequences of the reference corresponding to the fragmentation sites;
generating, using the plurality of fragmentation site contexts, a data structure encoding information about a distribution of fragmentation site contexts of the cfDNA sample; and
determining whether the subject has cancer using the data structure encoding information about the distribution of fragmentation site contexts of the cfDNA sample.

20. A non-transitory computer-readable storage medium storing instructions that, when executed by at least one computer hardware processor, cause the at least one computer hardware processor to perform a method for determining whether a subject has cancer by analyzing fragmentation in a cell-free deoxyribonucleic acid (cfDNA) sample obtained from the subject, the method comprising:
accessing sequencing data, the sequencing data previously obtained from sequencing the cfDNA sample, the sequencing data comprising a plurality of reads of cfDNA fragments;
aligning the plurality of reads to a reference;
identifying, using results of aligning the plurality of reads to the reference, fragmentation sites of the cfDNA sample and nucleotide subsequences of the reference corresponding to the fragmentation sites;
determining fragmentation site contexts of the cfDNA sample using the nucleotide subsequences of the reference corresponding to the fragmentation sites;
generating, using the plurality of fragmentation site contexts, a data structure encoding information about a distribution of fragmentation site contexts of the cfDNA sample; and
determining whether the subject has cancer using the data structure encoding information about the distribution of fragmentation site contexts of the cfDNA sample.
